(19) United States
(12) Patent Application Publication
(10) Pub. No.: US 2019/0192657 A1
(43) Pub. Date: Jun. 27, 2019

(54) NANO-SYSTEMS FOR THERAPY AND/OR DIAGNOSIS AND/OR THERAPY MONITORING AND/OR THERANOSTICS OF DISEASE

(71) Applicants: COSMOPHOS LTD, Thessaloniki (GR); UNIVERSIDAD AUTÓNOMA DE MADRID, Madrid (ES); KØBENHAVNS UNIVERSITET, København K (DK); UNIVERSITY OF EASTERN FINLAND, Kuopio (FI)

(72) Inventors: Panagiotis TROHOPOULOS, Thessaloniki (GR); Jørn Bolstad CHRISTENSEN, Frederiksberg (DK); Tomás TORRES-CEBADA, Madrid (ES); Seyed Moein MOGHIMI, Durham (GB); Seppo Pasi Antero YLÄ-HERTTUALA, Kuopio (FI); Mario FICKER, Frederiksberg (DK); Linping WU, Thornaby Stockton-on-Tees (GB); María MEDEL GONZÁLEZ, Madrid (ES); Petri Ilmari MÄKINEN, Kuopio (FI)

(21) Appl. No.: 16/099,035

(22) PCT Filed: May 3, 2017
(86) PCT No.: PCT/EP2017/060579

§ 371 Date: Nov. 5, 2018

(30) Foreign Application Priority Data
May 5, 2016 (EP) 16168476.6

(51) Int. Cl. A61K 41/00; A61K 49/00; A61P 35/00; A61P 9/10

(52) U.S. Cl. CPC A61K 41/0071; A61K 49/0036; A61P 9/10; A61K 49/0093; A61P 35/00; A61K 49/0054

(57) ABSTRACT

There is disclosed a composition comprising self-assembled nano-particles, the nano-particles comprising dendrimers having a phthalocyanine covalently bound to the periphery thereof. The composition is useful in the therapy and/or diagnosis and/or therapy monitoring and/or theranostics of a lesion of a tissue.

### FIELD OF THE INVENTION

The present invention relates to nano-systems comprising dendrimers and phthalocyanines. The nano-systems of the invention are of particular utility as therapeutic and/or diagnostic and/or therapy monitoring and/or theranostic (therapeutic and diagnostic and therapy-monitoring) nano-systems. The nano-systems may be used in, without limitation, endovascular photodynamic therapy and/or endovascular fluorescence-based imaging and/or endovascular real-time and follow-up therapy monitoring and/or endovascular theranostics (endovascular photodynamic therapy and endovascular fluorescence-based imaging and endovascular real-time and follow-up therapy monitoring) of, in particular, atherosclerotic cardiovascular diseases. The nano-systems may also be of utility in relation to other diseases.

### BACKGROUND OF THE INVENTION

Atherosclerotic cardiovascular diseases are the leading cause of human death and morbidity worldwide. Atherosclerosis is the predominant and most lethal arterial wall disease and is characterized by focal/regional/diffuse arterial lesions—the so-called atherosclerotic plaques—with thickening of the innermost layer of the artery, causing arterial stenosis. Atherosclerosis can affect any artery in the body, principally the large-sized elastic and the medium-sized muscular arteries. As a result, different atherosclerotic cardiovascular diseases may develop, depending on which arteries are affected by atherosclerosis:

coronary artery disease of the heart may lead to angina and myocardial infarction, known as heart attack; carotid artery disease may lead to brain stroke; peripheral artery disease may lead to claudication and gangrene; renal artery disease may lead to arterial hypertension and renal failure; and so on.

In atherosclerotic plaques, extracellular lipid droplets, cellular debris, and degraded extracellular matrix form a core region, called the necrotic core, which is surrounded by a cap of a collagen-rich matrix, foam cells, macrophage cells, and smooth muscle cells, called the fibrous cap. Although advanced atherosclerotic lesions may lead to ischemic symptoms as a result of progressive narrowing of the arterial lumen, acute and severe cardiovascular events, like heart attacks and brain strokes, mainly (>85% of all cases) result from the rupture or erosion of atherosclerotic plaques which are non-occlusive/non-flow-limiting and which cause in the majority of cases <50% stenosis of the arterial lumen and are clinically asymptomatic—the so-called “vulnerable” plaques.

Atherosclerotic plaque rupture is the leading cause of cardiovascular thrombosis and subsequent clinical manifestations, while plaque erosion is less frequent. Blood exposure of prothrombotic material from the necrotic core of ruptured/eroded plaque (oxLDL, phospholipids, tissue factor, platelet-adhesive matrix molecules, etc) disrupts hemostasis. When such pathologic processes overwhelm the regulatory mechanisms of hemostasis, thrombin is excessively formed endovascularly, initiating thrombosis. Platelets have a central role in cardiovascular thrombosis. They adhere to the sub-endothelial matrix after endothelial damage, and then aggregate with each other to form a prothrombotic surface that promotes clot formation and subsequently vascular occlusion. Thrombotic occlusion of a coronary artery of the heart results in acute myocardial infarction (heart attack), thrombotic occlusion of a carotid artery results in acute brain stroke, thrombotic occlusion of a peripheral artery results in gangrene, and so on.

Current strategies to fight the consequences of atherosclerotic cardiovascular diseases are orientated either towards the promotion of a healthy lifestyle (smoking cessation, balanced nutrition, exercise) and pharmacological treatment of “systemic” risk factors (dyslipidemia, arterial hypertension, hyperglycemia, etc), or towards late pharmacological strategies including thrombolysis and late interventional strategies including catheter-based balloon angioplasty (PCl/PTA) without or with “stent” (Drug Eluting Stent—DES, or Bare Metal Stent—BMS) placement, surgery (Coronary Artery Bypass Graft—CABG—surgery, or vascular surgery), or combinations of the above for the treatment of clinically symptomatic, occlusive atherosclerotic plaques (causing >70% arterial luminal stenosis). However, these clinically symptomatic, occlusive atherosclerotic plaques are responsible for only 14% of all heart attacks and strokes, the remaining 86% being due to clinically asymptomatic, non-occlusive/non-flow-limiting atherosclerotic plaques (those causing <70% arterial luminal stenosis, and in the majority of the cases even <50% arterial luminal stenosis)—so-called “vulnerable plaques”—which currently cannot be detected and properly treated. For acute cardiovascular events, prompt revascularization is indicated in order to save valuable tissue from necrosis. In acute myocardial infarction (heart attack), in acute brain stroke, and in gangrene, revascularization by using thrombolytics and/or balloon angioplasty/surgery immediately restores blood flow, thereby limiting heart/brain/limbs damage. However, these therapies need improvement for increased efficacy and decreased side effects (e.g. intracranial/gastrointestinal hemorrhage, “stent” restenosis/thrombosis, etc).

Unfortunately, a large number of poorly treated patients suffering from atherosclerotic cardiovascular diseases, and a huge number of the world's population with asymptomatic atherosclerotic cardiovascular diseases exist today, and they urgently need novel therapeutic and diagnostic modalities for long-term survival, substantially decreased mortality and morbidity rates, and quality of life. Despite the existing therapeutic arsenal in medicine, the incidence of cardiovascular clinical events still remains dramatically high, and for this reason atherosclerotic cardiovascular diseases are the leading cause of human death and morbidity worldwide. This demonstrates that currently there are major diagnostic and therapeutic gaps in the management of atherosclerotic cardiovascular diseases between screening and prevention on the one hand, and emergency diagnostic and treatment modalities on the other. In view of the severe and widespread mortality and morbidity associated with atherosclerotic cardiovascular diseases, there is an urgent unmet medical need for new approaches for early diagnosis, effective targeted therapy, therapy monitoring, and theranostics to tackle this major health issue.

Photodynamic therapy (PDT) is an emerging therapy, which in principle requires three interacting elements: 1) a light-activatable compound, the so-called photosensitizer; 2) light of appropriate wavelengths; and 3) tissue oxygen. Upon exposure of a photosensitizer to specific wavelengths of light, it becomes activated from a ground state to a singlet excited state, which in turn undergoes intersystem crossing to a triplet excited state. As the photosensitizer returns to the ground state, it releases energy, which is transferred to the surrounding tissue oxygen to generate reactive oxygen species (ROS), such as singlet oxygen (¹O₂) and free radicals. These ROS mediate cellular toxicity of targeted cells, inducing apoptosis (i.e. non-inflammatory programmed cell death). PDT has been investigated extensively in the laboratory for decades, and for over 30 years in the clinical environment. The approach is non-invasive or minimally invasive, enables accurate targeting, and repeated administration without total-dose limitations. The current clinical applications of PDT include the treatment of acne, non-melanoma skin cancer, head and neck cancer, Barrett's esophagus, and wet macular degeneration. Recently, endovascular PDT has emerged as a very promising therapeutic modality for the therapy of atherosclerotic cardiovascular diseases and restenosis after injury (like “stent” restenosis), and in short-term studies has shown efficacy in limiting atherosclerotic plaque inflammation in animal models. Additionally, endovascular PDT has proven safe and well tolerated in phase-I clinical trials for atherosclerotic heart disease patients and for patients with atherosclerotic peripheral artery disease. Photodynamic therapy simultaneously reduces plaque inflammation and promotes repopulation of plaques with a smooth muscle cell (SMC)-rich stable plaque cell phenotype, while reducing disease progression. These early healing responses suggest that endovascular PDT is a very promising therapeutic modality for the therapy of atherosclerotic cardiovascular diseases, coronary artery disease, acute coronary syndromes, carotid artery disease, strokes, and peripheral artery disease. The use of light in the so-called Biological Near-infrared (NIR) Window (650 nm-900 nm) enables endovascular NIR Photodynamic Therapy (endovascular NIR PDT) for highest blood and tissue penetration by the light and increased therapeutic efficacy through blood, without the need for arterial occlusion. Unfortunately, clinical trials on endovascular PDT for the therapy of atherosclerotic cardiovascular diseases in patients are limited. One of the main problems in photodynamic therapy, limiting the use of many potent photosensitizers, is the difficulty in preparing pharmaceutical formulations that enable their parenteral (IV) administration. Due to their low water solubility, very potent hydrophobic photosensitizers, like phthalocyanines, cannot be injected intravenously. Additionally, the targeted delivery of potent photosensitizers to atherosclerotic plaques is a very challenging problem.

In vivo fluorescence imaging is an emerging diagnostic modality, which in principle requires two interacting elements: 1) a light-activatable compound, the so-called fluorophore; and 2) light of appropriate wavelengths. Upon exposure of a fluorophore to specific wavelengths of light, it becomes activated from a ground state to a singlet excited state, and as the fluorophore returns to the ground state it emits fluorescence, usually at longer wavelengths. This emission can be visualized by appropriate sensors, enabling in vivo fluorescence imaging. In vivo fluorescence imaging has accelerated scientific discovery and development in the life sciences as it enables labeling of specific biochemical components and the visualization of biological processes. Endovascular fluorescence-based imaging has recently emerged as a very promising diagnostic modality of atherosclerotic cardiovascular diseases, and has focused on imaging components associated with atherosclerotic plaque inflammation achieving molecular imaging, by using either endovascular fluorescence imaging alone or in combination with: (a) intravascular ultrasound imaging (IVUS) and/or (b) optical coherence tomography imaging (OCT) and/or (c) photoacoustics (optoacoustics) imaging (PA or OA) and/or (d) near-infrared spectroscopy imaging (NIRS). During endovascular fluorescence-based imaging, the diagnostic endovascular catheter illuminates the blood vessel wall and collects the subsequent fluorescence. Early implementations were performed in 1D (one-dimensional) scans of the blood vessels along their length. 2D (two-dimensional) endovascular fluorescence-based imaging was later achieved by performing 360-degrees rotation in addition to endovascular catheter pullback, and was recently successfully demonstrated in vivo in atherosclerotic rabbit animal models. The use of light in the so-called Biological Near-infrared (NIR) Window (650 nm-900 nm) enables in vivo endovascular 2D NIR fluorescence-based molecular imaging (endovascular 2D NIRF-based molecular imaging) for highest blood and tissue penetration by the light and deep tissue imaging through blood, without considerable reduction in sensitivity and without the need for arterial occlusion, by using either endovascular 2D NIRF imaging alone or in combination with: (a) intravascular ultrasound imaging (IVUS) and/or (b) optical coherence tomography imaging (OCT) and/or (c) photoacoustics (optoacoustics) imaging (PA or OA) and/or (d) near-infrared spectroscopy imaging (NIRS).

Due to their low water solubility, very efficient hydrophobic fluorophores cannot be injected intravenously. Additionally, the targeted delivery of efficient fluorophores to the atherosclerotic plaques is a very challenging problem.

The use of nanotechnology for medical purposes (nanomedicine) has grown exponentially over the past few decades. Although the domain originally focused on anticancer therapy, recent advances have pointed to the tremendous potential of nanomedicine in the therapy and diagnosis of atherosclerotic cardiovascular diseases. The use of nano-systems in the therapy, diagnosis, therapy monitoring, and theranostics of atherosclerotic cardiovascular diseases has emerged as a very promising strategy for efficient targeted drug delivery, achieving several advantages which include: (i) the improved delivery of poorly water-soluble drugs; (ii) the targeted delivery of drugs by avoiding the reticuloendothelial system, and utilizing the enhanced permeability and retention effect (EPR effect), and the active tissue-specific targeting; (iii) the transcytosis of drugs across epithelial/endothelial barriers; (iv) the delivery of macromolecule drugs to intracellular sites of action; (v) the co-delivery of two or more drugs or therapeutic modalities for combined therapy; (vi) the visualization of sites of drug delivery by combining therapeutic agents with imaging moieties, and (vii) the real-time and follow-up read of the in vivo efficacy of a therapeutic agent. In this respect, various nano-particle types have already been successfully utilized in medicine as nanocarriers, including dendrimers.

Dendrimers are uniquely monodispersed compounds characterized by a structure built from a core by repetitive branching. This molecular architecture leads to molecules that rapidly grow to nanometer dimensions and are comparable to globular proteins with respect to size and molecular weight. The dendritic architecture gives rise to some characteristic properties: a large number of surface groups, interior voids (depending on the type of branch cell unit), low viscosity, good adherence to surfaces etc. The large number of surface groups has been utilized in applications such as enhancing sensitivity in bioassays, boosting binding of carbohydrates to cell surfaces and for inhibition of viral infections through blocking of viral receptors by multivalent display of aryl sulfonates. Dendrimers can also be used as nano-particle platforms, where a number of different molecules, ligands, reporter groups are confined in space through covalent bonding to the surface of the dendrimer. One example of the use of such a construct is the targeting dendrimer reported by Tsien and coworkers (“Activatable cell penetrating peptides linked to nano-particles as dual probes for in vivo fluorescence and MR imaging of proteases”, E. S. Olson, T. Jianga, T. A. Aguilera, Q. T. Nguyen, L. G. Ellies, M. Scadeng, R. Y. Tsien, PNAS 107, 4311-4316 (2010)), in which a PAMAM-dendrimer was used for assembling a fluorescence label, a MR-imaging agent and a peptide for dual imaging of tumors. The peptide tail on the dendrimer was converted into a targeting peptide upon contact with enzymes specific for the type of cancer in question, effectively labeling the tumor and aiding surgical removal.

The physical properties of dendrimers such as viscosity and adherence have been utilized in areas such as ink for ink-jet printing, in cosmetics, and in medicinal uses such as increasing adhesion of carbohydrates. There is a substantial literature available on dendrimers, their synthesis and properties, as exemplified by the following references:

a) “Dendrimers, new molecular tools in medicine and biotechnology” U. Boas, P. Heegaard, J. B. Christensen (Royal Society of Chemistry 2006);

b) “Dendrimers, Dendrons and Dendritic Polymers: From Discovery to Applications” D. A. Tomalia, U. Boas, J. B. Christensen (Cambridge University Press 2012);

c) “Dendrimers as therapeutic agents: a systematic review” V. Gajbhiye, V. K. Palanirajan, R. K. Tekade, N. K. Jain, Journal of Pharmacy and Pharmacology 61, 989-1003 (2009),

d) “Dendrimers and dendritic polymers in drug delivery” E. R. Gillies, J. M. J. Fréchet, Drug Discovery Today, 10, 35-43 (2005);

e) “Dendrimers Designed for Functions: From Physical, Photophysical, and Supramolecular Properties to Applications in Sensing, Catalysis, Molecular Electronics, Photonics, and Nanomedicine” D. Astruc, E. Boisselier, C. Ornelas, Chemical Reviews 110, 1857-1959 (2010).

As a result of their unique nano-structural features, dendrimers are already used—commercially or in clinical trials—in a wide variety of nanomedicine products including:

1) cardio diagnostics (Stratus; Siemens, Germany);

2) gene transfection vectors (Superfect; Qiagen, Germany);

3) cardiovascular surgery/ophthalmology (Adherus®; HyperBranch, USA); and

4) anti-virals/microbicides (VivaGel; Starpharma, Australia).

VivaGel-based anti-virals/microbicides active against HIV, genital herpes, bacterial vaginosis, etc, are currently in Phase-III clinical trials.

Photosensitizers are generally classified as either porphyrinoid or non-porphyrinoid derivatives. Among non-porphyrinoid photosensitizers, neutrally charged hypocrellin, squaraine and BODIPY derivatives, or cationic compounds such as chalcogenopyrylium, phenothiazinium and benzo[a]phenothiazinium dyes (which include methylene blue and toluidine blue) have been the predominant focus. However, due to the limited potency and various side effects associated with many of them, the development of non-porphyrinoid photosensitizers for application in medicine has lagged considerably behind that of porphyrinoid photosensitizers. Indeed, none have yet been approved for clinical use, and some have now been abandoned.

The porphyrinoid derivatives are further classified as first, second and third generation photosensitizers. First-generation photosensitizers include hematoporphyrin derivative (HpD) and porphimer sodium (Photofrin). A number of second-generation photosensitizers, like phthalocyanines, have been developed to alleviate certain problems associated with first-generation photosensitizers, such as prolonged skin photosensitization and suboptimal tissue penetration. These second-generation photosensitizers are chemically pure, absorb light at longer wavelengths, and cause significantly less skin photosensitization post-treatment. In addition, second-generation photosensitizers must be at least as efficient as Photofrin, the current gold standard for PDT. Second-generation photosensitizers bound to nanocarriers in order to become water soluble for parenteral (IV) administration, and for targeted accumulation within selective tissues are referred to as third-generation photosensitizers, and currently represent an active research area in the field.

Among porphyrinoid photosensitizers, porphimer sodium (Photofrin), palladium-bacteriopherophorbide (Tookad), NPe6, motexafin lutetium (Antrin, Lutrin or Lu-Tex) and phthalocyanines have been clinically approved or are currently under non-clinical or clinical investigation.

Phthalocyanines constitute one of the most promising families of the second-generation porphyrinoid photosensitizers with intrinsic fluorescence. Phthalocyanines are a group of photosensitizers/fluorophores structurally related to porphyrins. They present a number of properties that make them ideal PDT/fluorescence compounds.

Phthalocyanines are robust and very versatile molecules with a strong absorption at 670-770 nm (ε˜10⁵ M⁻¹ cm⁻¹). They yield high singlet oxygen production and long-lived fluorescence. Studies using the silicon phthalocyanine Pc 4 (λ_{ex/em}=675/690 nm), both in vitro and in vivo studies and also in a successfully completed Phase-I clinical trial for the treatment of cutaneous neoplasms, are so far the most promising (Baron, E. D. et al, Laser. Surg. Med. 2010, 42, 728-735).

General aspects of photosensitizers/fluorophores, and of phthalocyanines in particular, are collected in many monographs and scientific articles, such as:

(a) McKeown, N. B., Phthalocyanine Materials, Cambridge University Press, Cambridge, 1998;

(b) Dolmans, D. G. J.; Fukumura, D.; Jain, R. K.; Nat. Rev. Cancer 2003, 3, 380-387;

(c) Ormond, A. B., Freeman, H. S.; Materials 2013, 6, 817-840;

(d) Master, A., Livingston, M.; Sen Gupta, A.; J. Control. Release 2013, 168, 88-102;

(e) Menon, J.U., Jadeja, P., Tambe, P., Vu, K., Yuan, B., Nguyen, K. T., Theranostics 2013, 3, 152-166;

(f) Kadish, K. M., Smith K. M., Guilard, R., Handbook of Porphyrin Science, World Scientific, Singapore, 2013;

amongst others.

However, third-generation photosensitizers with intrinsic fluorescence (i.e. second-generation photosensitizers with intrinsic fluorescence bound to nanocarriers) have never been used for the therapy and/or diagnosis and/or therapy monitoring and/or theranostics of atherosclerotic cardiovascular diseases. This is highly desired but very challenging. Targeting moieties for atherosclerotic plaques have been suggested, most of which come from studies in the field of oncology. Indeed, there are commonalities between cancer and atherosclerotic disease with respect to the basic molecular and cellular mechanisms underlying neovascularization and inflammation.

RGD peptides have been successfully used for targeting cancer (Dijkgraaf, I. et al., Eur J Nucl Med Mol Imaging 2011), and atherosclerosis, combining molecular imaging with MRI and targeted drug delivery in rabbit models of atherosclerosis (Winter, P. M., Lanza, G. M. et al, Arterioscler Thromb Vasc Biol. 2006; 26:2103-2109). Cyclic RGD peptides bind to αᵥβ₃ integrin, a transmembrane molecule which is upregulated in neovascular endothelial cells and macrophage/foam cells within atherosclerotic plaques. Interference with integrin-mediated attachment of endothelial cells to the extracellular matrix protein fibronectin, results in anoikis of these cells.

LyP-1 is a cyclic nonapeptide which specifically binds to p32 in cancer lymphatics and cancer cells. Under physiological conditions p32 is a mitochondrial protein while under pathophysiological conditions the protein is highly overexpressed and presented on cell surfaces (Hamzaha, J. et al, PNAS 2011). Injection of fluorescent LyP-1 in atherosclerotic mice resulted in homing of the peptide to plaques, especially macrophages and foam cells, but also arterial luminal endothelial cells.

Another approach has made use of the presence in atherosclerotic plaques of apoptotic cells which expose phosphatidylserines. It has been proposed that especially in unstable atherosclerotic plaques, more apoptosis occurs and, conversely, that apoptosis contributes to plaque instability. As specifically in apoptotic cells phosphatidylserines are exposed, phosphatidylserine-binding peptides have been selected using phage display technology and such peptides have been utilized to visualize atherosclerotic plaques in mouse models of atherosclerosis (Burtea el al, Mol Pharm. 2009). From a therapeutic point of view, targeting of already apoptotic cells would obviously not be effective.

Other specific markers of atherosclerotic plaques have been identified also, including p32, interleukin-4 receptor (IL-4R) (Hong, J. et al, Cell Mod Med 2008), stabilin-2 (Young-Lee, J. et al, Contr Rel 2011), VCAM-1, and macrophage scavenger receptor A (Gough, Ylä-Herttuala, et al, ATVB 1999). These markers are expressed in neovascular endothelial cells and/or macrophage/foam cells within atherosclerotic plaques, but also frequently on luminal endothelial cells and smooth muscle cells in the fibrous cap and the tunica media. Therapeutic targeting of the latter cell population may be dangerous due to possible destabilization of an otherwise stable plaque. Targeting of the luminal endothelial cell layer is also unwanted, as endothelial cell death in the arterial lumen may result in thrombosis.

Plexin D1 is expressed on neovasculature and macrophages in cancer and inflammatory disorders. Antibodies (VHHs) against plexin D1 have been shown to accumulate in cancer neovasculature in mouse models of cancer (Roodink, I., Leenders, W. P. et al, Cancer Res 2005; Roodnik, .I, Leenders, W. P. et al, Cancer 2009, 9: 297; Roodnik, I., Leenders, W. P. et al, Lab Invest 2009, 90, 61-67), but targeting of atherosclerotic plaques via plexin D1 has never been attempted. Currently available VHHs against plexin D1 are of moderate affinity (Ka=20-30 nM).

Targeting moieties which specifically bind to macrophage/foam cells & neovascular endothelial cells within atherosclerotic plaques, whilst avoiding the luminal endothelial cells, and the smooth muscle cells of the fibrous cap and of the tunica media, are highly desired but are not yet available.

### SUMMARY OF THE INVENTION

According to a first aspect of the invention, there is provided a composition comprising self-assembled nano-particles, the nano-particles comprising dendrimers having a phthalocyanine covalently bound to the periphery thereof.

In a second aspect, the invention provides a composition comprising self-assembled nano-particles, the nano-particles comprising dendrimers associated with peripherally-substituted phthalocyanines, as defined herein.

The compositions of the invention are referred to generally herein as “nano-systems”.

The invention also provides a method for therapy and/or diagnosis and/or therapy monitoring and/or theranostics of a lesion of a tissue in a subject,
which method comprises the steps of
(a) identifying a subject having or suspected of having the lesion;
(b) administering to the subject a composition according to the invention;
(c) causing or allowing nano-particles present in said composition to accumulate upon and/or within the lesion, if present;
(d) exposing the tissue to electromagnetic radiation so as to cause the nano-particles to produce one or more of fluorescence, reactive oxygen species, heat, an optical signal and an acoustic signal.

For use in diagnosis and/or therapy monitoring and/or theranostics, the method may further comprise the step of detecting fluorescence, an optical signal and/or an acoustic signal produced by the nano-particles, such fluorescence, optical signal and/or acoustic signal being indicative of the presence, site and/or condition of the lesion.

When used therapeutically, the nano-particles may produce reactive oxygen species and/or heat, in such a manner as to bring about the death of cells within the lesion and/or the passivation of the lesion and/or the stabilization of the lesion and/or the regression of the lesion and/or the therapy of the lesion.

In a related aspect, the invention provides a composition according to the first or second aspects of the invention, for use in therapy and/or diagnosis and/or therapy monitoring and/or theranostics of a lesion of a tissue.

The dendrimers may be any suitable dendrimers, but most preferably the dendrimers are PAMAM dendrimers.

The dendrimers are preferably dendrimers of the first generation or higher, and more particularly are dendrimers of the third generation or higher.

The periphery of the dendrimer may be chemically modified to render the dendrimers more biocompatible, for example less immunogenic or toxic.

The phthalocyanine may be present in free base form or as a metal- or metalloid-complex. The phthalocyanine is a preferably a peripherally-substituted phthalocyanine, and most preferably is a zinc phthalocyanine.

An important benefit of the nano-systems of the invention is that they have been found to accumulate in atherosclerotic plaques and/or other lesions, without the need for conjugation with other targeting moieties, and so are effective delivery vehicles for PDT agents (ie the phthalocyanine component of the nano-particles) to those atherosclerotic plaques and/or other lesions. Thus, whilst in some embodiments the nano-particles may be conjugated or otherwise associated with targeting moieties such as those discussed herein, in other embodiments such tissue/cell-targeting moieties are unnecessary and the compositions do not contain any such tissue/cell-targeting moieties.

The compositions of the invention can be administered by a variety of routes, including without limitation parenterally (e.g. intravenously). The compositions therefore enable the targeted delivery of the phthalocyanines, which are potent photosensitizers and fluorophores, to atherosclerotic plaques and/or other lesions.

Without wishing to be bound by theory, it is believed that when the nano-systems of the first aspect of the invention are dispersed in an aqueous medium, the phthalocyanine moieties covalently bound to the periphery of a dendrimer, being highly hydrophobic, tend to become intercalated within a neighbouring dendrimer. This leads to self-assembly of the dendrimers to form nano-particles. Free phthalocyanine that is present may become entrapped within the hydrophobic interior of a dendrimer and/or free phthalocyanines may occupy the spaces between self-assembled dendrimers that make up the nano-particles. Similarly, in nano-systems of the second aspect of the invention, the phthalocyanine molecules, whilst not necessarily covalently bound to the dendrimers, nonetheless are believed to function as bridges between dendrimers, leading to self-assembly of the nano-particles.

### BRIEF DESCRIPTION OF THE DRAWINGS

FIGS. 1A-L show the size distribution profile of nano-systems according to the invention, as determined by nano-particle tracking analysis (NTA).

FIGS. 2A-S are plots of relative viability versus nano-system concentration for cells of mouse macrophage cell line RAW 264.7, after exposure to various concentrations of nano-systems of Examples 1-19 and illumination with light from an LED (ie PDT), showing the in vitro therapeutic efficacy of those nano-systems.

FIGS. 3A-C show fluorescence microscopy images demonstrating the in vivo accumulation of nano-systems of Examples 14, 13 and 7 respectively in mouse atherosclerotic plaques. The nuclei are stained with DAPI and appear as bright dots; the nano-system is fluorescent and appears as the continuously bright regions within the plaques.

FIGS. 4A-E show the in vivo accumulation of nano-systems of Examples 4, 1, 3, 8 and 9 respectively in mouse atherosclerotic plaques. The nuclei are stained with DAPI and appear as bright dots; the nano-system is fluorescent and appears as the more continuously bright regions within the plaques.

FIGS. 5A-C show the in vivo accumulation of nano-systems of Examples 14, 13 and 7 respectively in rabbit atherosclerotic plaques. The nuclei are stained with DAPI and appear as bright dots; the nano-system is fluorescent and appears as the continuously bright regions within the plaques.

FIGS. 6A-D show the in vivo accumulation of a nano-system of Examples 1, 3, 8 and 9 respectively in rabbit atherosclerotic plaques. The nuclei are stained with DAPI and appear as bright dots, the nano-system is fluorescent and appears as the continuously bright regions within the plaques.

FIGS. 7A-D show the in vivo co-localization of nano-system of Example 1 with the foam/macrophage cells in rabbit atherosclerotic plaques. FIG. 7A shows the fluorescence emitted from the nano-system, FIG. 7B shows a-SMA staining of smooth muscle cells, FIG. 7C shows RAM-11 staining of foam/macrophage cells and FIG. 7D shows the images of FIGS. 7A-C overlaid on one another.

FIG. 8 shows the in vivo targeted accumulation of a nano-system of Example 8 in rabbit atherosclerotic plaques. Such a nano-system does not accumulate in the endothelium or in the media (smooth muscle cells) or in the adventitia of the arterial wall. The arced parallel lines are the endothelium/smooth muscle cell (SMC) layers, the grey dots are the nano-system and the bright dots are the nuclei.

FIG. 9 shows that a nano-system of Example 4 does not accumulate in the healthy arterial wall in a balloon-injured New Zealand white (NZW) rabbit model of atherosclerosis.

FIGS. 10A and B demonstrate a substantial decrease of the intraplaque foam/macrophage cells (RAM-11 staining) in (B) treated compared to (A) non-treated rabbit atherosclerotic plaques using a nano-system of Example 8 which accumulated in rabbit atherosclerotic plaques.

FIGS. 11A and B show a close up of intraplaque foam/macrophage cells (RAM-11 staining) in (B) treated compared to (A) non-treated rabbit atherosclerotic plaques using a nano-system of Example 8 which accumulated in rabbit atherosclerotic plaques.

FIGS. 12A and B demonstrate a substantial increase of the intraplaque synthetic smooth muscle cells (α-SMA staining) in a layer structure arrangement in (B) treated compared to (A) non-treated rabbit atherosclerotic plaques using a nano-system of Example 8 which accumulated in rabbit atherosclerotic plaques.

FIG. 13 is a Transmission Electron Microscope (TEM) image of a nano-system according to Example 8.

FIG. 14 is a plot of relative viability versus nano-system concentration for cells of breast cancer cell line MCF-7, after exposure to various concentrations of the nano-system of Example 11 and illumination with light from an LED (ie PDT), showing the in vitro therapeutic efficacy of that nano-system.

FIG. 15 shows the in vivo co-localization of a nano-system of Example 8 with the macrophage cells in NZW-rabbit inflamed skeletal muscle. FIG. 15A shows the fluorescence emitted from the nano-system, FIG. 15B shows RAM-11 staining of macrophage cells, FIG. 15C shows DAPI staining of cell nuclei, and FIG. 15D shows the images of FIGS. 15A-C overlaid on one another.

FIG. 16 shows the in vivo co-localization of a nano-system of Example 8 with the macrophage cells in NZW-rabbit inflamed skin. FIG. 16A shows the fluorescence emitted from the nano-system, FIG. 16B shows RAM-11 staining of macrophage cells, FIG. 16C shows DAPI staining of cell nuclei, and FIG. 16D shows the images of FIGS. 16A-C overlaid on one another.

### DETAILED DESCRIPTION OF THE INVENTION

Definitions

As used herein, and unless indicated otherwise or the context requires otherwise:

“alkyl” as a group or part of a group means, unless otherwise specified, an aliphatic hydrocarbon group which may be straight or branched, and which, unless otherwise specified, may have from 1 to 20 carbon atoms.

“alkoxy” as a group or part of a group means a group —OR, where R is an alkyl group.

“cycloalkyl” means a saturated or bicyclic ring system of 3 to 10 carbon atoms.

“aryl” as a group or part of a group denotes: (i) an optionally substituted monocyclic or multicyclic aromatic carbocyclic moiety of 6 to 14 carbon atoms, such as phenyl or naphthyl; or (ii) an optionally substituted partially saturated multicyclic aromatic carbocyclic moiety in which an aryl and a cycloalkyl or cycloalkenyl group are fused together to form a cyclic structure.

“heteroaryl” as a group or part of a group denotes: (i) an optionally substituted aromatic monocyclic or multicyclic organic moiety of 5 to 10 ring members in which one or more of the ring members is/are element(s) other than carbon, for example nitrogen, oxygen or sulfur; or (ii) an optionally substituted partially saturated multicyclic heterocarbocyclic moiety in which a heteroaryl and a cycloalkyl or cycloalkenyl group are fused together to form a cyclic structure.

“metalloid” means a chemical element that is a non-metal but which has properties that are in one or more relevant respects comparable to those of metals. Examples of metalloids include silicon, germanium, boron, arsenic, antimony and tellurium.

Nano-Particles

The composition according to the first aspect of the invention comprises self-assembled nano-particles, the nano-particles comprising dendrimers having a phthalocyanine covalently bound to the periphery thereof. In nano-systems of the second aspect of the invention, peripherally-substituted phthalocyanine molecules are not, or at least are not all, covalently bound to the dendrimers, but nonetheless are believed to function as bridges between dendrimers, leading to self-assembly of the nano-particles.

The term “nano-particles” as used herein means particles having dimensions in the range 1 to 500 nm. In general, in a nano-system of the invention at least 50% w/w (dry basis) of the composition is in the form of nano-particles in that range, or 60%, 70%, 80%, 90%, 95% or 100% w/w. In particular embodiments, at least 50% w/w (dry basis) of the composition is in the form of nano-particles having sizes in the range 1 to 200 nm, or 60%, 70%, 80%, 90%, 95% or 100% w/w. At least 50% w/w, or 60%, 70%, 80%, 90%, 95% or 100% w/w of the composition may be in the form of nano-particles having sizes in the range 1 to 100 nm.

The compositions of the invention are preferably polydisperse, comprising a mixture of assemblies of dendrimer dimers, trimers and/or higher multimers.

The self-assembled nano-particles are preferably at least 5 nm in size. The self-assembled nano-particles preferably have a mean hydrodynamic size in the range 20 to 200 nm, more particularly 40 to 200 nm. More preferably the nano-particles have a mean hydrodynamic size in the range 40 to 150 nm, or 50 to 100 nm.

The nano-particles may be any suitable shape but typically the nano-particles have shapes that are spherical, oblate, oblate spheroid or oblate ellipsoidal.

Where, as in the first aspect of the invention, phthalocyanine moieties are covalently attached to the periphery of the dendrimers, the composition may further comprise phthalocyanines non-covalently associated with the self-assembled nano-particles. Without wishing to be bound by theory, it is believed that a phthalocyanine may be non-covalently associated with the dendrimers by being solubilised within a single dendrimer or by occupying the space between two or more dendrimers as those dendrimers self-assemble to form a nano-particle.

The nano-particles may have any suitable loading of phthalocyanine. Preferably, the composition according to the invention comprises from 0.1 to 20% w/w of phthalocyanine on a dry weight basis. The number of phthalocyanines present per dendrimer may vary considerably and may be dependent on the generation of the dendrimer, and therefore the size of the dendrimer. Typically the stoichiometric ratio of phthalocyanine:dendrimer will be between 0.1:1 and 20:1, preferably between 0.1:1 and 10:1, more preferably between 0.5:1 and 8:1. For G3 dendrimers, the stoichiometric ratio of phthalocyanine:dendrimer will typically be between 0.1:1 and 3:1, for G4 dendrimers between 1:1 and 5:1, and for G5 dendrimers between 2:1 and 10:1.

In the compositions of the invention, the nano-particles are preferably not conjugated to tissue/cell-targeting moieties. It has surprisingly been found that the nano-particles of the invention are capable of passive targeted accumulation at desired biological targets without the need for tissue/cell-targeting moieties. Thus, the compositions of the invention may be free of targeting moieties against cell surface receptors and antigens (e.g. folate receptor, prostate-specific membrane antigen protein, cell adhesion molecules such as integrins, cadherins and selectins), intracellular components and organelles (e.g. cytoplasmic proteins and enzymes, mitochondria, nucleus) and extracellular components (e.g. proteoglycans of extracellular matrix, metalloproteinases). These targeting moieties, without limitation, include small molecules (e.g. folate, 2-[3-(1, 3-dicarboxy propyl)-ureido] pentanedioic acid, mono- and oligosaccharides, nucleic acids), peptides (e.g. RGD peptides, phage-derived peptides, cell penetrating peptides, LyP-1 cyclic nonapeptide, phosphatidylserine-binding peptides, mitochondrial targeting peptides, nuclear localisation sequence peptide), single-stranded RNA or DNA (i.e. aptamers), proteins (e.g. hormones, soluble forms of receptors), polyclonal antibodies, monoclonal antibodies, antibody fragments (e.g. Fab, F(ab′)2, Fab2, trispecific Fab3, bispecific diabody, trispecific triabody, scFv, minibody, V-NAR), and nanobodies (e.g. VHHs).

Dendrimers

As discussed above, dendrimers are monodispersed compounds characterised by a structure built from a core by repetitive branching out from the core. Dendrimers may be classified by their generation, which refers to the number of repeated branching cycles that are performed during synthesis. For example, if the branching reactions to synthesise the dendrimer are performed onto the core molecule three times, the resulting dendrimer is considered a third generation dendrimer.

As used herein, the term “periphery” of the dendrimer refers to the portion of the dendrimer that corresponds to the highest numbered generation of the dendrimer and, where the relevant phthalocyanine or functional groups are referred to as being attached to the periphery of the dendrimer, they are attached either directly or indirectly, for example via a linker group, to the highest numbered generation of the dendrimer. As dendrimers generally have a globular form, the periphery may also be thought of as the surface of the dendrimer. Attachment of the phthalocyanine molecule to the periphery of the dendrimer is to be contrasted with attachment of the phthalocyanine to sites internal to the dendrimer and to the use of phthalocyanines as dendrimer cores on which dendritic structures are built up.

The dendrimers used in the present invention may be any suitable dendrimer. Preferably the dendrimers are selected from the group consisting of polyamidoamine (PAMAM) dendrimers, polypropyleneimine (PPI) dendrimers, poly-lysine dendrimers, phosphorus dendrimers and polyester dendrimers. Most preferably the dendrimers are PAMAM dendrimers.

The dendrimers used in the present invention preferably included dendrimers of the first generation or higher. Most preferably the dendrimers include dendrimers of the third generation or higher. The dendrimers may be entirely dendrimers of the first generation or higher, or of the third generation or higher.

As a function of increasing size or generation (Gn), examples of PAMAM dendrimers (PD) may be represented by the formulae:

((CH₂)ₙN(CH₂CH₂CONH(CH₂) NRₐR_{b})₂)₂ PD-G0:

((CH₂)ₙN(CH₂CH₂CONH(CH₂)ₘN(CH₂CH₂CONH(CH₂)ₘNRₐR_{b})₂)₂)₂ PD-G1:

((CH₂)ₙN(CH₂CH₂CONH(CH₂)ₘN(CH₂CH₂CONH(CH₂)ₘN(CH₂CH₂CONH(CH₂)ₘ NRₐR_{b})₂)₂)₂)₂ PD-G2:

((CH₂)ₙN(CH₂CH₂CONH(CH₂)ₘN(CH₂CH₂CONH(CH₂)ₘN(CH₂CH₂CONH(CH₂)ₘ N(CH₂CH₂CONH(CH₂)mNRₐR_{b})₂)₂)₂)₂)₂ PD-G3:

((CH₂)ₙN(CH₂CH₂CONH(CH₂)ₘN(CH₂CH₂CONH(CH₂)ₘN(CH₂CH₂CONH(CH₂)ₘ N(CH₂CH₂CONH(CH₂)ₘN(CH₂CH2CONH(CH₂)mNRₐR_{b})₂)₂)₂)₂)₂)₂ PD-G4:

((CH₂)ₙN(CH₂CH₂CONH(CH₂)ₘN(CH₂CH₂CONH(CH₂)ₘN(CH₂CH₂CONH(CH₂)ₘ N(CH₂CH₂CONH(CH₂)ₘN(CH₂CH2CONH(CH₂)ₘN(CH₂CH₂CONH(CH₂)ₘ NRₐR_{b})₂)₂)₂)₂)₂)₂)₂ PD-G5:

((CH₂)ₙN(CH₂CH₂CONH(CH₂)ₘN(CH₂CH₂CONH(CH₂)ₘ N(CH₂CH₂CONH(CH₂)ₘN(CH₂CH₂CONH(CH₂)ₘN(CH₂CH₂CONH(CH₂)mN(CH₂CH2C ONH(CH₂)ₘN(CH₂CH₂CONH(CH₂)ₘNRₐR_{b})₂)₂)₂)₂)₂)₂)₂)₂ PD-G6:

((CH₂)ₙN(CH₂CH₂CONH(CH₂)ₘN(CH₂CH₂CONH(CH₂)ₘN(CH₂CH₂CONH(CH₂)ₘ N(CH₂CH₂CONH(CH₂)ₘN(CH₂CH₂CONH(CH₂)ₘN(CH₂CH₂CONH(CH₂)ₘN(CH₂CH2C ONH(CH₂)ₘN(CH₂CH₂CONH(CH₂)ₘNRₐR_{b})₂)₂)₂)₂)₂)₂)₂)₂)₂ PD-G7:

((CH₂)ₙN(CH₂CH₂CONH(CH₂)ₘN(CH₂CH₂CONH(CH₂)ₘN(CH₂CH₂CONH(CH₂)ₘ N(CH₂CH₂CONH(CH₂)ₘN(CH₂CH₂CONH(CH₂)ₘN(CH₂CH₂CONH(CH₂)ₘN(CH₂CH₂CO NH(CH₂)ₘN(CH₂CH₂CONH(CH₂)ₘN(CH₂CH₂CONH(CH₂)ₘNRₐR_{b})₂)₂)₂)₂)₂)₂)₂)₂)₂)₂ PD- G8:

((CH₂)ₙN(CH₂CH₂CONH(CH₂)ₘN(CH₂CH₂CONH(CH₂)ₘN(CH₂CH₂CONH(CH₂)ₘ N(CH₂CH₂CONH(CH₂)ₘN(CH₂CH₂CONH(CH₂)ₘN(CH₂CH₂CONH(CH₂)ₘ N(CH₂CH₂CONH(CH₂)ₘN(CH₂CH₂CONH(CH₂)ₘN(CH₂CH2CONH(CH₂)ₘ N(CH₂CH₂CONH(CH₂)ₘNRₐR_{b})₂)₂)₂)₂)₂)₂)₂)₂)₂)₂)₂ PD-G9:

((CH₂)ₙN(CH₂CH₂CONH(CH₂)ₘN(CH₂CH₂CONH(CH₂)ₘN(CH₂CH₂CONH(CH₂)ₘ N(CH₂CH₂CONH(CH₂)ₘN(CH₂CH₂CONH(CH₂)ₘN(CH₂CH₂CONH(CH₂)ₘN(CH₂CH₂CO NH(CH₂)ₘN(CH₂CH₂CONH(CH₂)ₘN(CH₂CH₂CONH(CH₂)ₘN(CH₂CH2CONH(CH₂)ₘN(CH₂CH₂CONH(CH₂)ₘNRₐR_{b})₂)₂)₂)₂)₂)₂)₂)₂)₂)₂)₂)₂ PD-G10:

wherein
n and m independently take values from 2 to 16,
Rₐ and R_{b} groups are independently H, an alkyl group of from 1-16 carbon atoms, or —COR, where R represents an alkyl group from 1 to 16 carbon atoms, cycloalkyl, aryl, heteroaryl, hydroxyalkyl ω-amidoalkyl, or ω-alkoxyalkyl,
or some or all of the Rₐ and R_{b} groups together with the nitrogen atom to which they are attached form a cyclic structure:

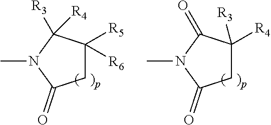

wherein
p and q independently take values from 0 to 5, and
in relation to these structures R₃, R₄, R₅, and R₆ are independently hydrogen, —COOR₇, or —CONHR₇,
in which R₇ may be hydrogen or an alkyl group of from 1 to 6 carbon atoms.

The compositions of the invention may comprise mixtures of two or more dendrimers of different chemical classes. More commonly, however, the compositions comprise dendrimers of a single chemical class, most preferably PAMAM dendrimers.

The dendrimers may all be dendrimers of the same generation, eg G3, G4, G5 or a higher generation. Alternatively, the dendrimers may be a mixture of two or more dendrimer generations, e.g. a mixture of two or more of G3, G4, G5 or higher generations.

Dendrimers with amino surface groups can be obtained from commercial sources or may synthesized according to the literature (e.g. D. A. Tomalia, H. Baker, J. Dewald, M. Hall, G. Kallos, S. Martin, J. Roeck, J. Ryder, P. Smith Polymer Journal 1985, 17, 117-32.).

Dendrimers with 4-carbomethoxy pyrrolidone surface groups may be synthesized as described in WO-A-2004/069878.

Surface Modification of Dendrimers

The properties of the dendrimers may be altered by modifying the periphery of the dendrimer with one or more surface chemical groups. The chemical groups may be chosen for example to increase the biocompatibility of the dendrimer or to increase the solubility of the dendrimer in a particular solvent, and/or to render the dendrimers less toxic, and in particular less immunogenic.

The surface chemical groups may, without limitation, be selected from the group consisting of amine, amide, carboxybetaine, sulfobetaine, triazoliumcarboxylate, phosphorylcholine, pyrrolidone, 2-amino-2-hydroxymethyl-propane-1,3-diol, hydroxyl, carboxyl, methoxy, ethoxy, 4-carbomethoxy pyrrolidone, poly(ethylene glycol), and any combination thereof.

In some embodiments the dendrimer has surface groups having the structure:

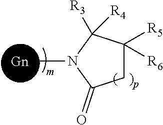

wherein
Gn represents a dendrimer of generation n;
m is from 1 to the maximum number of available surface groups on the dendrimer;
p is from 0 to 5; and
in relation to these structures R₃, R₄, R₅, R₆ are independently H, —COOR₇, —CONHR₇, in which R₇ is H, alkyl having 1 to 6 carbon atoms, or —C(CH₂OH)₃.

One example of the modification of a dendrimer surface with 4-carbomethoxy pyrrolidone groups (“pyrrolidone”) is depicted in the following scheme:

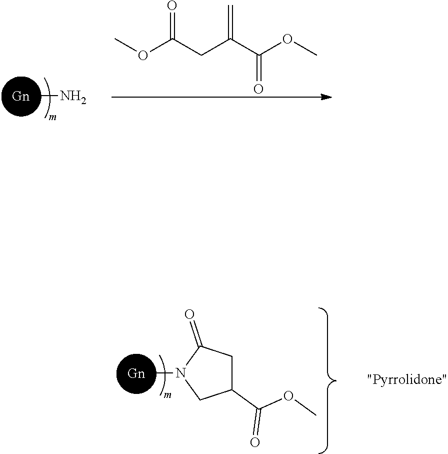

In other embodiments, the dendrimer displays surface groups having the structure:

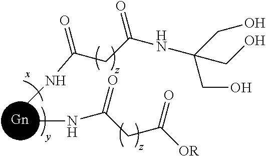

wherein
Gn represents a dendrimer of generation n;
x and y are independently from 1 to the maximum number of available surface groups on the dendrimer;
the z values are independently from 0 to 16; and
R is H or alkyl having 1 to 6 carbon atoms.

A diagrammatic representation of a dendrimer surface modification with a 4-(1,3-dihydroxy-2-(hydroxymethyl)propan-2-ylamino)-4-oxobutanamide (“Succinic acid linker+Tris(hydroxymethyl)aminomethane”), which is also referred to herein as a TRIS surface group, and with a 4-(amino)-4-oxobutanoate (“Succinic carboxylate surface”), which is also referred to herein as a carboxylate surface group is shown below.

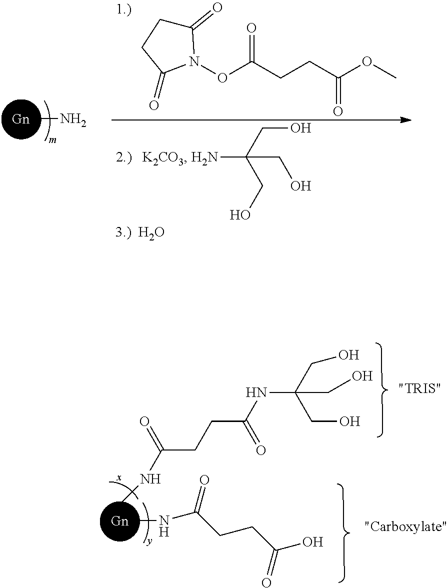

The result is a dendrimer with mixed surface groups, with synthetically variable ratios between carboxy and TRIS surface groups. These dendrimers are also referred to herein as carboxy/TRIS dendrimers.

Phthalocyanines

The phthalocyanine used in the present invention may be a compound of the general structure:

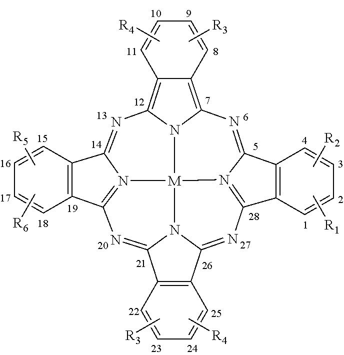

wherein
M represents a metal or metalloid atom,
in relation to this structure R₁, R₂, R₃, R₄, R₅ and R₆ are independently selected from the group consisting of hydrogen, —COOH, —C≡C—COOH, —CH═CHCOOH, an alkyl group having from 1 to 12 carbon atoms, —OR₇, —SR₇ or —NR₇R₈,
in which R₇ and R₈ independently represent H, an alkyl group having from 1 to 12 carbon atoms, or a phenyl group optionally substituted by one or more R₉ groups independently selected from the group consisting of an alkyl group having from 1 to 12 carbon atoms, —OR₁₀, —SR₁₀, and —NR₁₁R₁₂,
wherein R₁₀, R₁₁ and R₁₂ each independently represent H or an alkyl group having from 1 to 12 carbon atoms, or
one or more pairs of R₁ and R₂, R₃ and R₄, and R₅ and R₆ are attached to adjacent carbon atoms and together form, together with the ring to which they are attached, an aromatic fused ring system.

Where R₁, R₂, R₃, R₄, R₅ and R₆ are attached to adjacent carbon atoms and together form, together with the ring to which they are attached, an aromatic fused ring system, the compound may be a naphthalocyanine.

In other embodiments, the phthalocyanine may have the free base form:

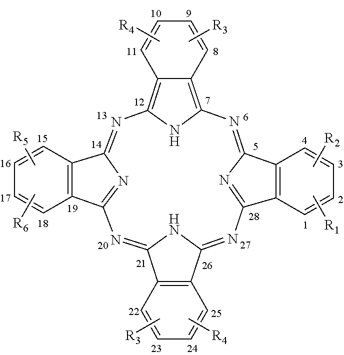

in which the groups R₁, R₂, R₃, R₄, R₅ and R₆ are as defined above.

Unsubstituted phthalocyanine, and many of its complexes, have very low solubility in many solvents. The phthalocyanine is therefore, preferably, a peripherally-substituted phthalocyanine.

By “peripherally-substituted” is meant that at least one of the groups R₁, R₂, R₃, R₄, R₅ and R₆ is other than hydrogen. Preferably, two, three or all four of the fused rings of the phthalocyanine structure carry at least one substituent.

Peripherally-substituted phthalocyanines are essentially planar or discotic in shape, which is believed to facilitate their intercalation within the dendrimers. The presence of peripheral substituents, on the other hand, is believed to prevent excessive self-aggregation of the phthalocyanine molecules.

In contrast, an “axially substituted” phthalocyanine carries substituents located axially relative to the plane of the phthalocyanine molecule on a metal or metalloid complexed by the phthalocyanine. Preferably the phthalocyanines of the present invention are peripherally- and not axially-substituted phthalocyanines.

The phthalocyanine may be complexed with any suitable metal or metalloid. Preferably the phthalocyanine is a zinc phthalocyanine. More preferably the phthalocyanine is a compound of the general formula ZnPc:

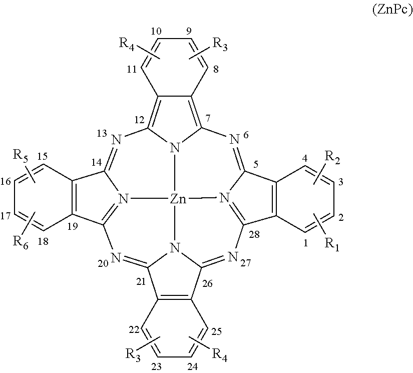

wherein
R₁ and R₂ are independently selected from the group consisting of

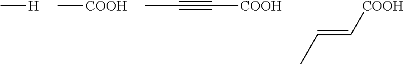

and
R₃, R₄, R₅ and R₆ are as defined above.

The carboxy-containing groups R₁ and R₂ may be located independently in any of the two central positions of the corresponding benzene ring, i.e. in positions 2 and/or 3 of compound ZnPc. In a preferred embodiment, the carboxy-containing groups R₁ and/or R₂ are carboxy groups.

The R₃, R₄, R₅ and R₆ groups on the isoindole rings may be located independently in any of the two central positions of each one of the corresponding benzene rings, i.e. in positions 9 and/or 10, 16 and/or 17, and 23 and/or 24 of the isoindole rings of compound ZnPc.

The R₃, R₄, R₅ and R₆ groups on the isoindole rings may be located independently in any of the two central positions of each one of the corresponding benzene rings, and preferably are tert-butyl groups.

In other embodiments, the R₃, R₄, R₅ and R₆ groups on the isoindole rings may be located independently in any of the two central positions of each one of the corresponding benzene rings, and are 2,6-diphenylphenoxy groups.

In another particular embodiment, the R₃ and R₄ groups located independently on any of the two central positions of each one of the corresponding benzene rings are tert-butyl or 2,6-diphenylphenoxy groups, while R₅ and R₆ groups located independently in any of the two central positions on the corresponding benzene ring are independently H, an alkyl group having from 1 to 12 carbon atoms, —OR₇, —SR_{S}, or —NR₇R₈, where R₇ and R₈ are as set out above.

Most preferably the phthalocyanine is a compound of the formula:

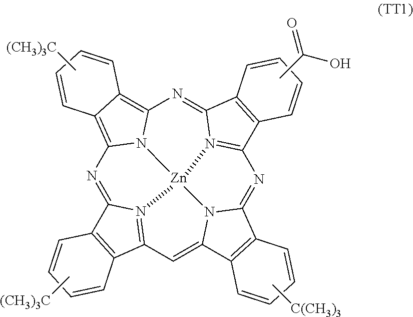

its regioisomers and mixtures thereof. Compounds of this structure are referred to herein as “TT1” and may be prepared as described in Angew. Chem. Int. Ed. 2007, 46, 8358-8362.

The term “regioisomers” refers to position isomers having the same functional group or substituent in different positions; regioisomers have the same molecular formula but often different chemical and physical properties.

Preferably the phthalocyanine is a compound selected from the group of regioisomers consisting of:
9,16,23-tri-tert-butyl-2-carboxy-5,28:14,19-diimino-7,12:21,26-dinitrilo-tetrabenzo[c,h,m,r][1,6,11,16]tetraazacycloeicosinato-(2⁻)-N²⁹,N³⁰,N³¹,N³² zinc (II);
9,16,24-tri-tert-butyl-2-carboxy-5,28:14,19-diimino-7,12:21,26-dinitrilo-tetrabenzo[c,h,m,r][1,6,11,16]tetraazacycloeicosinato-(2⁻)-N²⁹,N³⁰,N³¹,N³² zinc (II);
9,17,23-tri-tert-butyl-2-carboxy-5,28:14,19-diimino-7,12:21,26-dinitrilo-tetrabenzo[c,h,m,r][1,6,11,16]tetraazacycloeicosinato-(2⁻)-N²⁹,N³⁰,N³¹,N³² zinc (II);
9,17,24-tri-tert-butyl-2-carboxy-5,28:14,19-diimino-7,12:21,26-dinitrilo-tetrabenzo[c,h,m,r][1,6,11,16]tetraazacycloeicosinato-(2⁻)-N²⁹,N³⁰,N³¹,N³² zinc (II);
10,16,23-tri-tert-butyl-2-carboxy-5,28:14,19-diimino-7,12:21,26-dinitrilo-tetrabenzo[c,h,m,r][1,6,11,16]tetraazacycloeicosinato-(2⁻)-N²⁹,N³⁰,N³¹,N³² zinc (II);
10,16,24-tri-tert-butyl-2-carboxy-5,28:14,19-diimino-7,12:21,26-dinitrilo-tetrabenzo[c,h,m,r][1,6,11,16]tetraazacycloeicosinato-(2⁻)-N²⁹,N³⁰,N³¹,N³² zinc (II);
10, 17, 23-tri-tert-butyl-2-carboxy-5,28:14,19-diimino-7,12:21,26-dinitrilo-tetrabenzo[c,h,m,r][1, 6, 11, 16]tetraazacycloeicosinato-(2⁻)-N²⁹, N³⁰, N³¹, N³² zinc (II);
10,17,24-tri-tert-butyl-2-carboxy-5,28:14,19-diimino-7,12:21,26-dinitrilo-tetrabenzo[c,h,m,r][1,6,11,16]tetraazacycloeicosinato-(2⁻)-N²⁹,N³⁰,N³¹,N³² zinc (II);
and mixtures thereof.

Most preferably the phthalocyanine is a compound comprising a mixture of regioisomers 9(10), 16(17), 23(24)-tri-tert-butyl-2-carboxy-5,28:14,19-diimino-7,12:21,26-dinitrilo-tetra-benzo[c,h,m,r][1,6,11,16]tetraazacycloeicosinato-(2⁻)-N²⁹,N³⁰,N³¹,N³² zinc (II).

Carboxyphthalocyanines of formula ZnPc can be prepared following methods described in the state of the art (see for example, Angew. Chem. Int. Ed. 2007, 46, 8358-8362, Chem. Eur. J. 2009, 15, 5130-5137, Energy Environ. Sci., 2011, 4, 189-194, Chem. Sci., 2011, 2, 1145-1150, Angew. Chem. Int. Ed. Eng. 2012, 51, 4375-4378, Org. Biomol. Chem. 2013, 11, 2237-2240, and literature cited therein), and they can be either pure compounds (pure regioisomers) or mixtures of two or more regioisomers.

The compositions of the invention may comprise a phthalocyanine of a single chemical structure (including mixtures of regioisomers). Alternatively, the compositions of the invention may comprise mixtures of two or more chemically different phthalocyanines.

Conjugation of phthalocyanine to Dendrimer

The phthalocyanines may be covalently bound to the periphery of the dendrimer by any suitable means. Preferably, the at least one phthalocyanine is conjugated to the dendrimer by NHS-ester activation of a carboxy substituent on the phthalocyanine followed by coupling to a peripheral amine group on the dendrimer to form an amide linkage.

Other forms of conjugation chemistry may be used, which will be familiar to those skilled in the art. Without limitation, where the phthalocyanine carries one or more carboxylic acid groups, conjugation may be via any group on the dendrimer periphery that is capable of reacting with such a carboxyl group, e.g. hydroxyl groups, amine groups, or derivatives thereof. The phthalocyanine may be covalently bonded directly to the periphery of the dendrimer, as described in the immediately preceding paragraph. Alternatively, the phthalocyanine may be indirectly conjugated to the dendrimer, i.e. conjugated via a spacer group. Again, suitable spacer groups and methods of indirect conjugation will be familiar to those skilled in the art.

The following illustrations show schematically the structures of exemplary nano-particles, comprising dendrimer and (covalently and/or non-covalently) associated phthalocyanine. In these structures, it will be appreciated that the phthalocyanine moieties are shown on a somewhat reduced scale. It will also be appreciated that covalent attachment of a phthalocyanine may occur at any of the positions on the periphery of the dendrimer, and in general there will be a statistical distribution of attachment positions. Similarly, phthalocyanine molecules not covalently attached to the dendrimer may occupy any positions within the dendrimer, and indeed may generally be in a dynamic equilibrium with phthalocyanine molecules that are not entrapped within the dendrimer structure.

a) A covalent nano-system [Cov-D-(ZnPc)ₙ] having a 4-carbomethoxy pyrrolidone functionalised dendrimer surface and 2 ZnPc molecules covalently linked to the dendrimer.

b) A non-covalent nano-system [NonCov-D-(ZnPc)ₙ] having a 4-carbomethoxy pyrrolidone functionalised dendrimer surface and 2 ZnPc molecules non-covalently linked to a dendrimer.

c) A mixed covalent/non-covalent nano-system [Cov/NonCov-D-(ZnPc)ₙ] having a 4-carbomethoxy pyrrolidone functionalised dendrimer surface and 2 ZnPc molecules covalently linked to a dendrimer and 1 ZnPc non-covalently linked to a dendrimer.

d) A covalent nano-system [Cov-D-(ZnPc)n] having a carboxy/tris functionalised dendrimer surface and 3 ZnPc molecules covalently linked to the dendrimer.

e) A non-covalent nano-system [NonCov-D-(ZnPc)n] having a carboxy/tris functionalised dendrimer surface and 3 ZnPc molecules non-covalently linked to a dendrimer.

f) A mixed covalent/non-covalent nano-system [Cov/NonCov-D-(ZnPc)n] having a carboxy/tris functionalised dendrimer surface and 3 ZnPc molecules covalently linked to a dendrimer and 1 ZnPc non-covalently linked to a dendrimer.

Formulations of the Nano-Systems of the Invention

The compositions according to the invention may be formulated in any suitable dosage form, for example as a solution or suspension. Preferably the compositions are in a form suitable for injection. Such forms are typically solutions or dispersions, usually in an aqueous medium.

Preferably the composition is in the form of a solution or dispersion of the self-assembled nano-particles in an aqueous medium, or is a lyophilised material. Such a formulation allows for simple administration of the composition by injection, e.g. intravenous injection or intramuscular injection, or other suitable means. Lyophilised material may for example be reconstituted with water, saline solution or similar media prior to administration.

In other embodiments, the composition may be suitable for topical administration, e.g. being formulated as gels (water- or alcohol-based), creams or ointments containing nano-systems of the invention.

In other embodiments, the composition may be suitable for oral administration (per os), e.g. being formulated as tablets or capsules containing nano-systems of the invention.

In other embodiments, the composition may be suitable for direct administration to a lesion of a tissue by any suitable means.

Applications of the Nano-Systems

The compositions according to the invention are of use in a method for therapy and/or diagnosis and/or therapy monitoring and/or theranostics of a lesion of a tissue in a subject. Thus another embodiment of the invention relates to a method for therapy and/or diagnosis and/or therapy monitoring and/or theranostics of a lesion of a tissue in a subject,
which method comprises the steps of
(a) identifying a subject having or suspected of having the lesion;
(b) administering to the subject a composition according to the invention;
(c) causing or allowing nano-particles present in said composition to accumulate upon and/or within the lesion, if present;
(d) exposing the tissue to electromagnetic radiation so as to cause the nano-particles to produce one or more of fluorescence, reactive oxygen species, heat, an optical signal and an acoustic signal.

Without wishing to be bound by theory, it is believed that, by self-assembly of the dendrimers, the nano-particles are a suitable size that they are absorbed by, or accumulate upon, the lesions automatically without the requirement for any targeting moieties.

The method may further comprise the step of detecting fluorescence, an optical signal and/or an acoustic signal produced by the nano-particles, such fluorescence, optical signal and/or acoustic signal being indicative of the presence, site and/or condition of the lesion. This enables the signal emitted by the nano-particles to be used to identify the presence of lesions and their location and thus can be used as a method of diagnosis and/or therapy monitoring and/or theranostics.

Phthalocyanines are photoactive and, after irradiation by a light source, may produce fluorescence, reactive oxygen species, heat, an optical signal or an acoustic signal. Preferably the phthalocyanines are capable of producing fluorescence and/or reactive oxygen species after irradiation by a light source.

By producing fluorescence, once accumulated in the lesions, the phthalocyanines can be used to locate the lesions within the subject thus enabling diagnosis and are also therefore of use in directing targeted therapies.

In one embodiment, the phthalocyanines produce reactive oxygen species and/or heat, in such a manner as to bring about the death of cells within the lesion and/or the passivation of the lesion and/or the stabilization of the lesion and/or the regression of the lesion and/or the therapy of the lesion by photodynamic therapy (PDT). Hence the compositions of the invention may be of use in theranostics, ie combining diagnosis of a condition or conditions, usually through imaging, with therapy of the same condition and therapy monitoring. Reactive oxygen species act by damaging the targeted tissue and, by generating heat, localised hyperthermia may be induced.

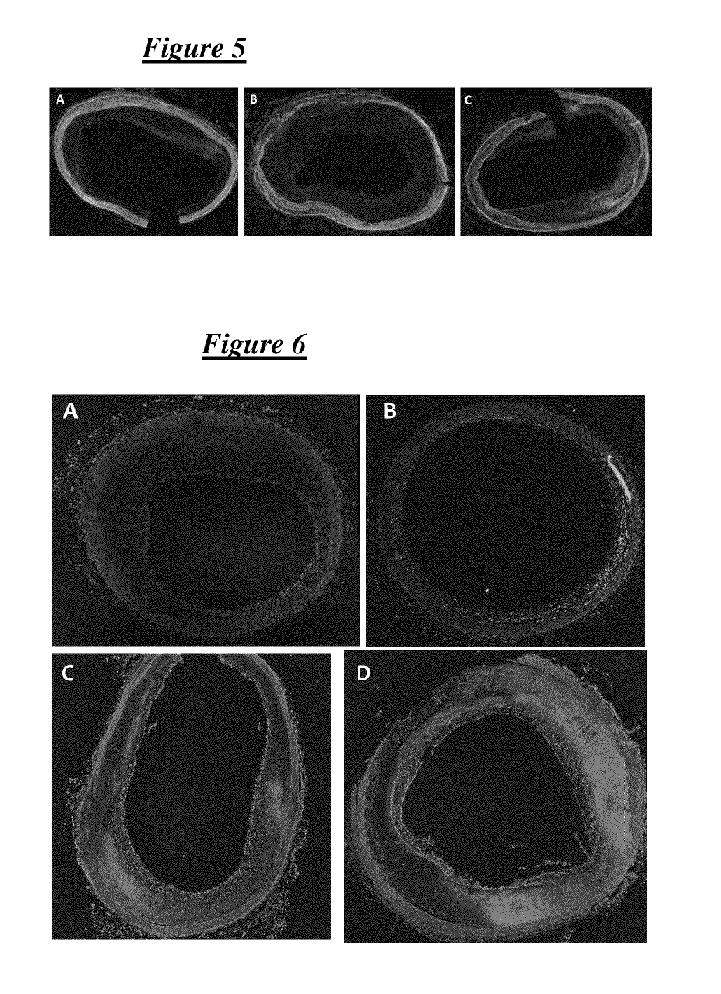

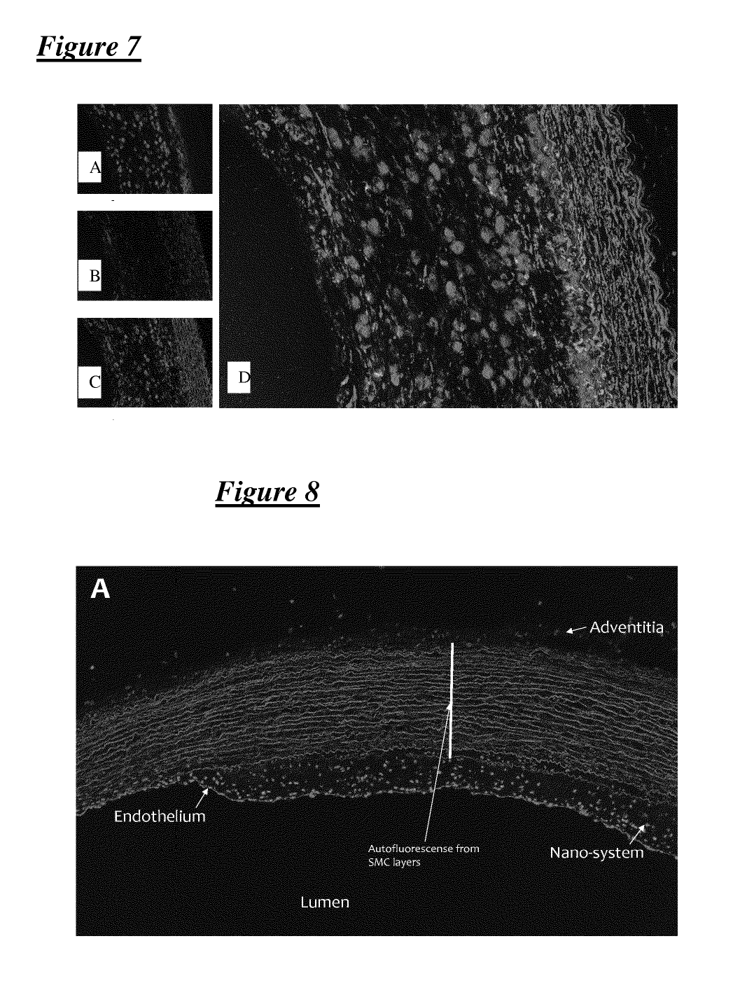

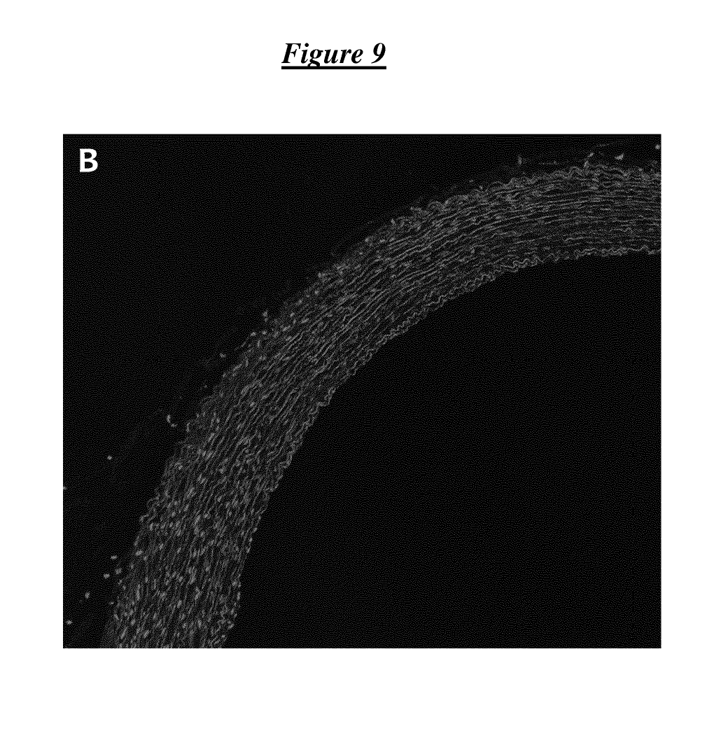

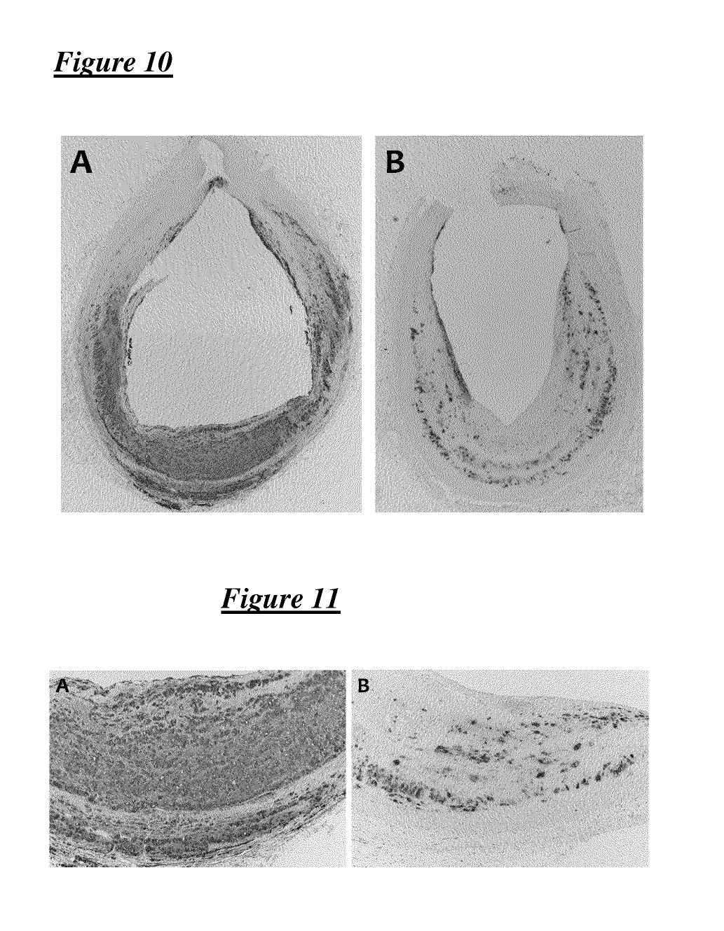

As demonstrated in FIGS. 10, 11 and 12, the compositions of the present invention are also suitable for monitoring the efficacy of a therapy. As the nano-particles fluoresce, they can be used to monitor the presence, and hence the treatment, of lesions during and after the therapeutic procedure. This can in turn be used to monitor the efficacy of the therapy both during the therapeutic procedure (real-time therapy monitoring) and at any time point after the therapeutic procedure (follow-up therapy monitoring).

The compositions of the invention may be administered by any suitable route. For example by oral, parenteral, intranasal, sublingual, rectal, transdermal, inhalation or insufflation routes, and direct administration to a lesion of a tissue. Preferably, the compositions are administered parenterally, most preferably by intravenous injection.

The nano-particles of the invention are caused or allowed to accumulate upon and/or within the lesion. This is most commonly brought about by means of a delay between the administration of the composition and subsequent activation. This delay provides sufficient time for the nano-particles to circulate within the subject and to accumulate upon and/or within the lesions. Typically, the delay may be from several minutes to several weeks or months, e.g. from 10 minutes to three months, or from 10 minutes to four weeks, or from 10 minutes to 2 weeks, 1 week, 48 hours, or 24 hours, or from 24 hours to three months, or from 24 hours to four weeks, or from 1 week to three months, or from 1 week to four weeks.

The compositions of the present invention are useful in treating a number of diseases within a subject. Without limitation, the compositions may be used for the therapy and/or diagnosis and/or therapy monitoring and/or theranostics of atherosclerosis, cancer and other conditions including, without limitation, inflammatory diseases such as inflammatory bowel disease, rheumatoid arthritis and autoimmune conditions, as well as infectious diseases and inflammation arising from infectious disease.

The methods and compositions of the invention are typically used for the therapy and/or diagnosis and/or therapy monitoring and/or theranostics of lesions of any tubular tissue, for example blood vessels, lymphatic vessels, respiratory tract, gastrointestinal tract, bile ducts, urinary tract or genital tract. Activation of the phthalocyanine photosensitizer may be brought about by means of a catheter, for example an optical fiber catheter or a side-firing and all-round emission optical fiber catheter, or the like introduced into the tubular tissue. The methods and compositions of the invention may, however, also be suitable for the therapy of solid tumours, for instance by topical application to skin tumours or by intra-operative direct administration to solid tumours of internal organs/tissues.

The invention will now be described in greater detail, by way of illustration only, with reference to the following Examples.

Abbreviations

ZnPc: Phthalocyanines of the general structure ZnPc, as defined herein

PT: Pyrrolidone-terminated

PD: PAMAM dendrimer

TT1: Phthalocyanines of the formula TT1, as defined herein

CTT: Carboxy/TRIS-terminated

AT: Amine-terminated

2,5-Dioxopyrrolidin-1-yl methyl succinate may be synthesized according to G. A. Digenis, B. J. Agha, K. Tsuji, M. Kato, M. Shinogi, J. Med. Chem. 1986, 29, 1468-1476.

### EXAMPLE 1

Covalent 4-carbomethoxy Pyrrolidone G3-PAMAM Dendrimer—ZnPc (TT1) Nano-System, having an Average of 1.0 ZnPc (TT1) Molecules Per Dendrimer

[Cov-PT-G3-PD-(TT1)_{1.0}]

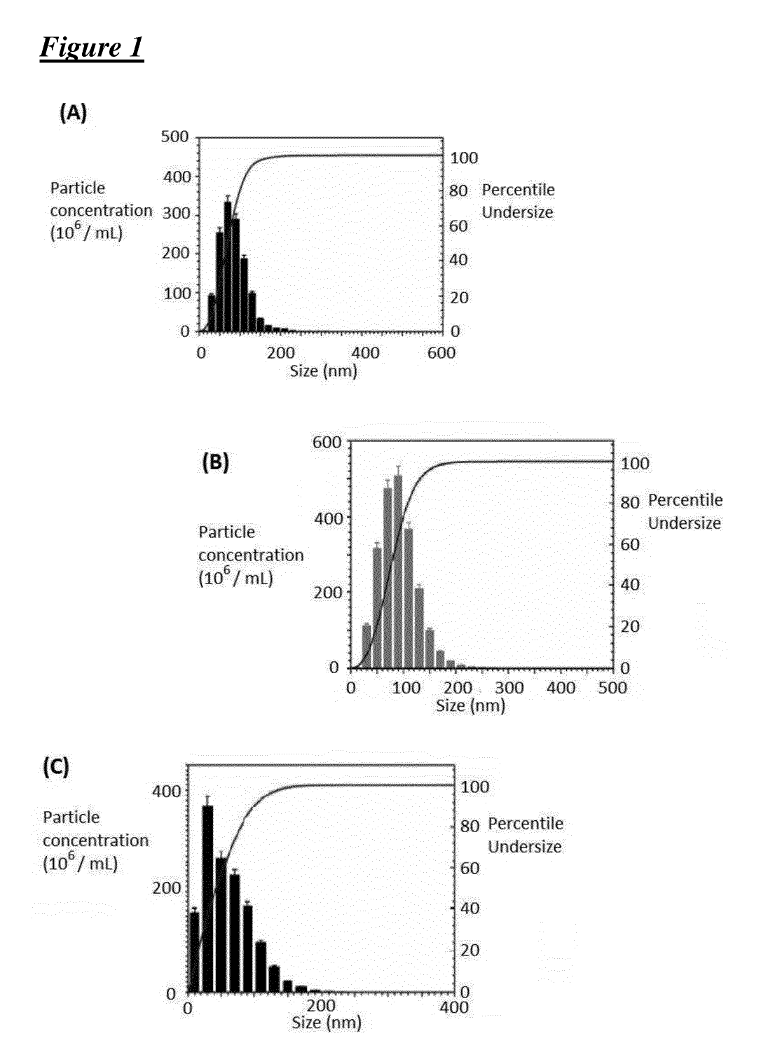

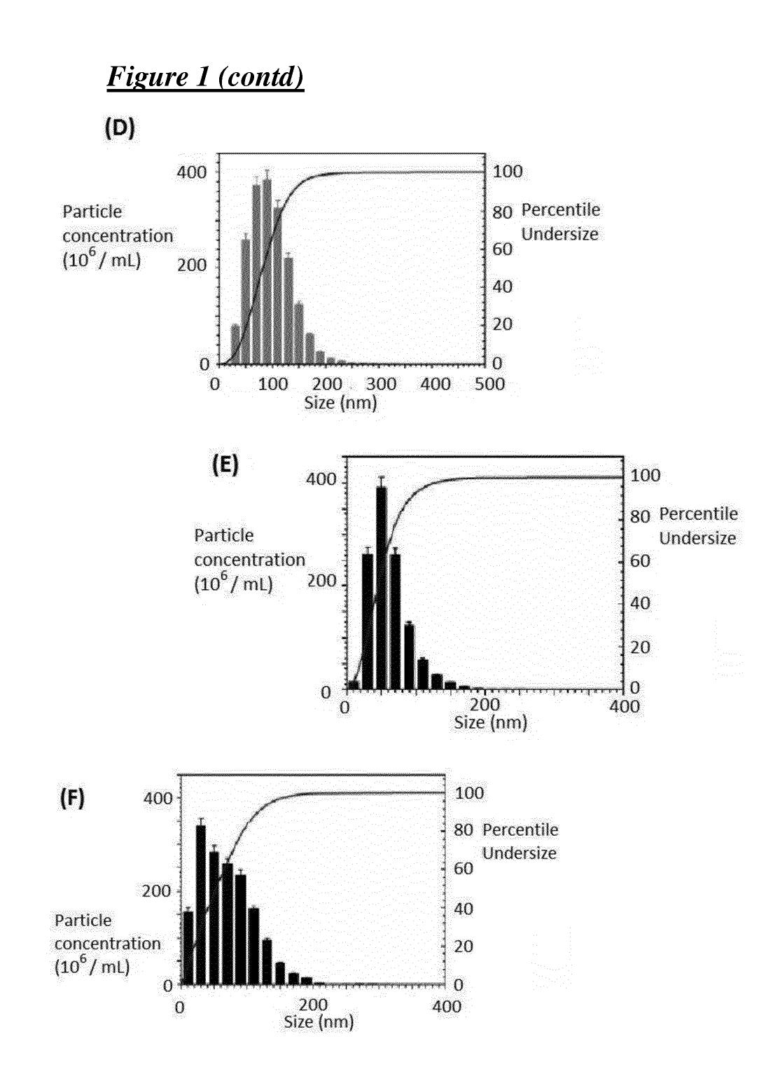

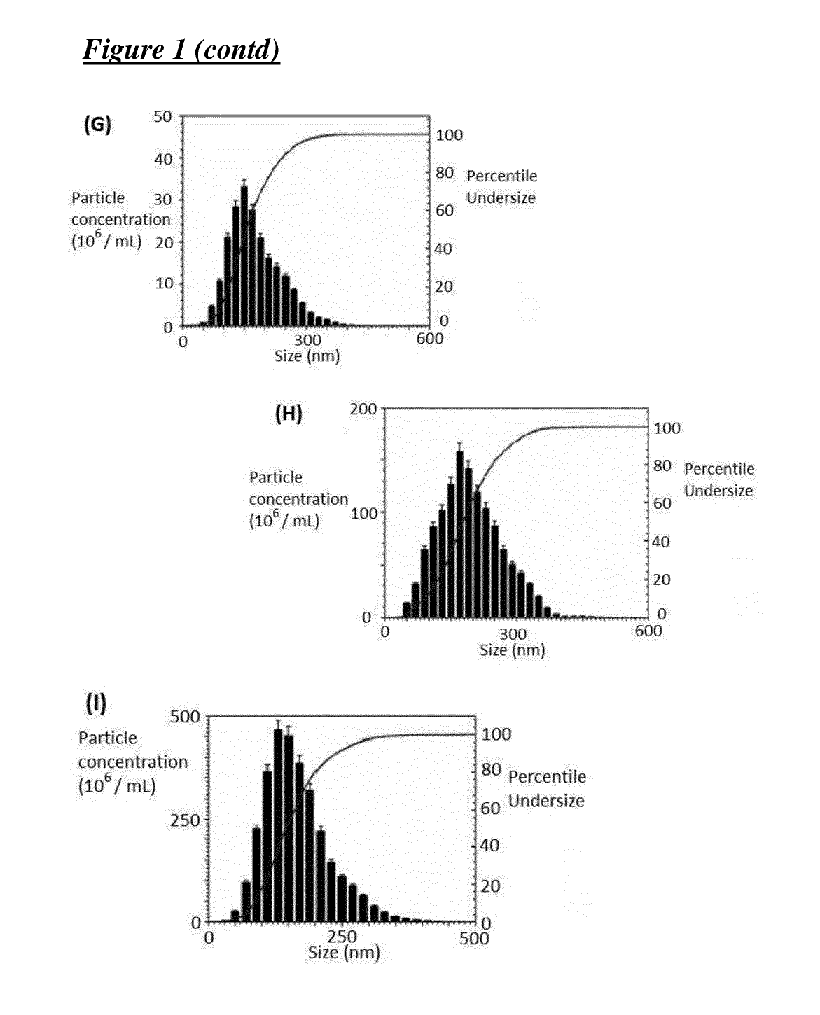

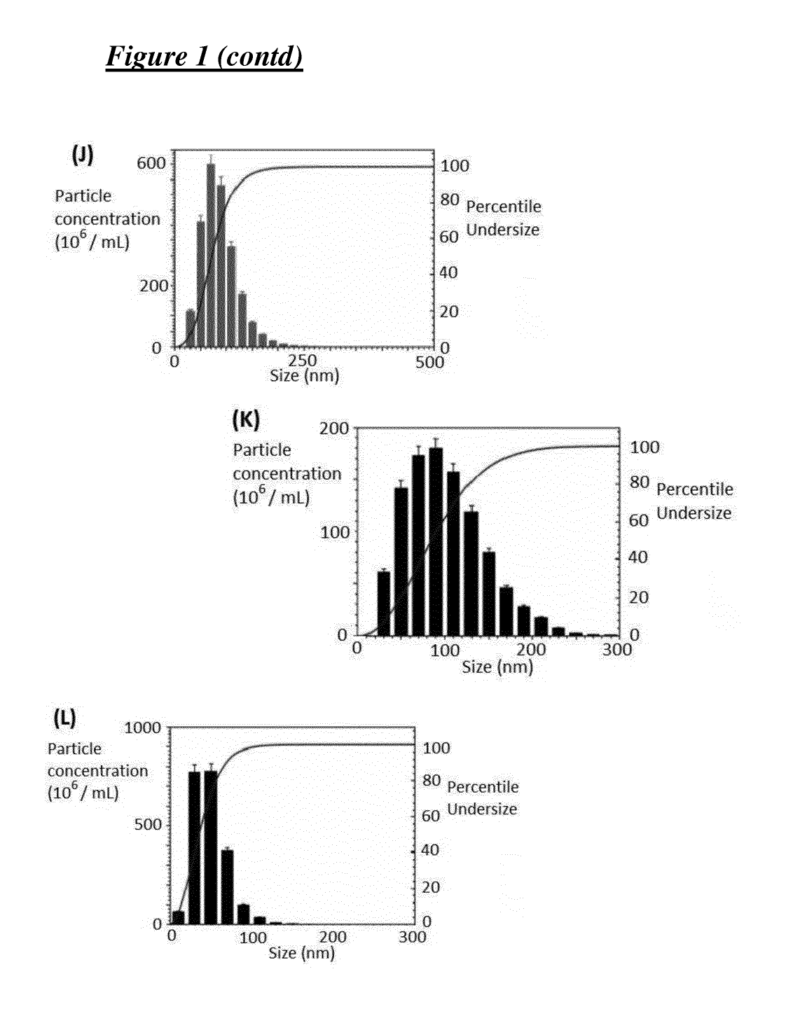

The activated ZnPc-NHS (TT1-NHS) ester was prepared by dissolving ZnPc (TT1, 28.8 mg) in dichloromethane (DCM) (2.5 mL). N-hydroxysuccinimide (4.25 mg) was dissolved in dimethyl sulfoxide (DMSO) (5 mL) and added to the ZnPc (TT1) solution followed by the addition of N,N′-dicyclohexylcarbodiimide (7.5 mg) to the reaction mixture. The reaction was stirred overnight and insoluble side products were removed from the reaction mixture by filtration, followed by a removal of the DCM solvent content under reduced pressure. The activated ZnPc-NHS (TT1-NHS) ester (dissolved in DMSO was added to a solution of G3-PAMAM (1,4-diaminobutane core) dendrimer (255 mg) in methanol (6 mL). The reaction was stirred 4 days, followed by a removal of insoluble side products by filtration. The dendrimer—ZnPc solution was then directly used for dendrimer surface functionalization without further purification. The PAMAM dendrimer solution from the previous reaction [ZnPc (TT1)—coupling] was taken and added to a solution of dimethyl itaconate (200 mg) dissolved in methanol. The solution was cooled with an ice bath during the addition. The reaction was stirred for four days. The final compound was purified by dialysis and filtration. After freeze-drying the dendrimer—ZnPc (TT1) nano-system was gained as a dark blue solid (260 mg). The structure was confirmed by NMR and the ZnPc (TT1) loading was measured by means of UV/Vis spectroscopy resulting in 1 ZnPc (TT1) molecules per dendrimer in average. The nano-system size was measured by nano-particle tracking analysis (NTA) and found to have 88±15 nm mean hydrodynamic size as shown in Table 1 and FIG. 1(A).

### EXAMPLE 2

Covalent 4-carbomethoxy pyrrolidone G3-PAMAM Dendrimer—ZnPc (TT1) Nano-System, having an Average of 1.4 ZnPc (TT1) Molecules Per Dendrimer

[Cov-PT-G3-PD-(TT1)_{1.4}]

The activated ZnPc-NHS (TT1-NHS) ester was prepared by dissolving ZnPc (TT1, 87 mg) in dichloromethane (5 mL). N-hydroxysuccinimide (12.7 mg) was dissolved in DMSO (10 mL) and added to the ZnPc (TT1) solution followed by the addition of N,N′-dicyclohexylcarbodiimide (22.6 mg) to the reaction mixture. The reaction was stirred overnight and insoluble side products were removed from the reaction mixture by filtration, followed by a removal of the DCM solvent content under reduced pressure. The activated ZnPc-NHS (TT1-NHS) ester dissolved in DMSO was added to a solution of G3-PAMAM [1,4-diaminobutane core) dendrimer (0.51 g, 73.5 μmol) in methanol (12 mL)]. The reaction was stirred 4 days, followed by a removal of insoluble side products by filtration. The dendrimer—ZnPc (TT1) solution was then directly used for dendrimer surface functionalization. The PAMAM dendrimer solution from the previous reaction was taken and added to a solution of dimethyl itaconate (401 mg) dissolved in methanol (2 mL). The solution was cooled with an ice bath during the addition. The reaction was stirred for four days. The final compound was purified by dialysis and filtration. After freeze-drying the dendrimer—ZnPc (TT1) nano-system was gained as a dark blue solid (640 mg). The structure was confirmed by NMR and the ZnPc (TT1) loading was measured by means of UV/Vis spectroscopy resulting in 1.4 ZnPc (TT1) molecules per dendrimer in average. The nano-system size was measured by nano-particle tracking analysis (NTA) and found to have 117±14 nm mean hydrodynamic size as shown in Table 1.

### EXAMPLE 3

Covalent Carboxylate/TRIS G3-PAMAM Dendrimer—ZnPc (TT1) Nano-System, Having an Average of 1.0 ZnPc (TT1) Molecules Per Dendrimer

[Cov-CTT-G3-PD-(TT1)_{1.0}]

The activated ZnPc-NHS (TT1-NHS) ester was prepared by dissolving ZnPc (TT1, 28.8 mg) in dichloromethane (DCM) (2.5 mL). N-hydroxysuccinimide (4.25 mg) was dissolved in dimethyl sulfoxide (DMSO) (5 mL) and added to the ZnPc (TT1) solution followed by the addition of N,N′-dicyclohexylcarbodiimide (7.5 mg) to the reaction mixture. The reaction was stirred overnight and insoluble side products were removed from the reaction mixture by filtration, followed by a removal of the DCM solvent content under reduced pressure. The activated ZnPc-NHS (TT1-NHS) ester dissolved in DMSO was added to a solution of G3-PAMAM (1,4-diaminobutane core) dendrimer (255 mg) in methanol (6 mL). The reaction was stirred 4 days, followed by a removal of insoluble side products by filtration. 2,5-Dioxopyrrolidin-1-yl methyl succinate (291 mg) (G. A. Digenis, B. J. Agha, K. Tsuji, M. Kato, M. Shinogi, J. Med. Chem. 1986, 29, 1468-1476) was added to the solution. The reaction mixture was stirred for two days and afterwards 2-amino-2-hydroxymethyl-propane-1,3-diol (185 mg) was added. Potassium carbonate was added in catalytic amounts to promote the reaction. After four days stirring at ambient temperature, the reaction was quenched with water to hydrolyze the unreacted methyl esters to carboxylic groups. The final compound was purified by dialysis and filtration. After freeze-drying the dendrimer—ZnPc (TT1) nano-system was gained as a dark blue solid (250 mg). The structure was confirmed by NMR (mixed dendrimer surface with approximately 29 Carboxylate and 1 TRIS dendrimer surface groups in average) and the ZnPc (TT1) loading was measured by means of UV/Vis spectroscopy resulting in 1 ZnPc (TT1) molecules per dendrimer in average. The nano-system size was measured by nano-particle tracking analysis (NTA) and found to have 91±10 nm mean hydrodynamic size as shown in Table 1 and FIG. 1(B).

### EXAMPLE 4

Covalent Carboxylate/TRIS G3-PAMAM Dendrimer—ZnPc (TT1) Nano-System, Having an Average of 1.4 ZnPc (TT1) Molecules Per Dendrimer

[Cov-CTT-G3-PD-(TT1)_{1.4}]

The activated ZnPc-NHS (TT1-NHS) ester was prepared by dissolving ZnPc (TT1, 87 mg) in dichloromethane (5 mL). N-Hydroxysuccinimide (12.7 mg) was dissolved in DMSO (10 mL) and added to the ZnPc (TT1) solution followed by the addition of N,N′-dicyclohexylcarbodiimide (22.6 mg) to the reaction mixture. The reaction was stirred overnight and insoluble side products were removed from the reaction mixture by filtration, followed by a removal of the DCM solvent content under reduced pressure. The activated ZnPc-NHS (TT1-NHS) ester dissolved in DMSO was added to a solution of G3-PAMAM (1,4-diaminobutane core) dendrimer (0.51 g) in methanol (12 mL). The reaction was stirred 4 days, followed by a removal of insoluble side products by filtration. The dendrimer-ZnPc (TT1) solution was then directly used for dendrimer surface functionalization without further purification. 2,5-Dioxopyrrolidin-1-yl methyl succinate (593 mg) (G. A. Digenis, B. J. Agha, K. Tsuji, M. Kato, M. Shinogi, J. Med. Chem. 1986, 29, 1468-1476) was added to the solution. The reaction mixture was stirred for two days and afterwards 2-amino-2-hydroxymethyl-propane-1,3-diol (370 mg) was added.

Potassium carbonate was added in catalytic amounts to promote the reaction. After four days stiffing at ambient temperature, the reaction was quenched with water to hydrolyze the unreacted methyl esters to carboxylic groups. The final compound was purified by dialysis and filtration. After freeze-drying the dendrimer—ZnPc (TT1) nano-system was gained as a dark blue solid (456 mg). The structure was confirmed by NMR (mixed dendrimer surface with approximately 29 Carboxylate and 1 TRIS dendrimer surface groups in average) and the ZnPc (TT1) loading was measured by means of UV/Vis spectroscopy resulting in 1.4 ZnPc (TT1) molecules per dendrimer in average. The nano-system size was measured by nano-particle tracking analysis (NTA) and found to have 86±5 nm mean hydrodynamic size as shown in Table 1.

### EXAMPLE 5

Covalent 4-carbomethoxy pyrrolidone G4-PAMAM Dendrimer—ZnPc (TT1) Nano-System, Having an Average of 3 ZnPc (TT1) Molecules Per Dendrimer

[Cov-PT-G4-PD-(TT1)₃]

ZnPc (TT1, 222 mg) was dissolved in dichloromethane (DCM) (25 mL). N-hydroxysuccinimide (33 mg) was dissolved in acetonitrile (10 mL) and added to the ZnPc (TT1) solution followed by the addition of N,N′-dicyclohexylcarbodiimide (58 mg) to the reaction mixture. The reaction was stirred overnight and insoluble side products were removed from the reaction mixture by filtration, followed by a removal of the solvent under reduced pressure. The ZnPc-NHS (TT1-NHS) ester was dissolved in DMSO (12 mL) and added to a solution of G4-PAMAM (1,4-diaminobutane core) dendrimer (0.50 g) in methanol (12 mL). The reaction was stirred overnight, followed by a removal of insoluble side products by filtration. The PAMAM dendrimer solution from the previous reaction was directly taken and added to a solution of dimethyl itaconate (373 mg) dissolved in methanol (2 mL). The solution was cooled with an ice bath during the addition. The reaction was stirred for four days. The final compound was purified by dialysis and filtration. After freeze-drying the dendrimer—ZnPc (TT1) nano-system was gained as a dark blue solid (450 mg). The structure was confirmed by NMR and the ZnPc (TT1) loading was measured by means of UV/Vis spectroscopy resulting in 3 ZnPc (TT1) molecules per dendrimer in average. The nano-system size was measured by nano-particle tracking analysis (NTA) and found to have 186±11 nm mean hydrodynamic size as shown in Table 1 and FIG. 1(G).

### EXAMPLE 6

Covalent Carboxylate/TRIS G4-PAMAM Dendrimer—ZnPc (TT1) Nano-System, Having an Average of 3 ZnPc (TT1) Molecules Per Dendrimer

[Cov-CTT-G4-PD-(TT1)₃]

ZnPc (TT1, 222 mg) was dissolved in dichloromethane (25 mL). N-hydroxysuccinimide (33 mg) was dissolved in acetonitrile (10 mL) and added to the ZnPc (TT1) solution followed by the addition of N,N′-dicyclohexylcarbodiimide (58 mg) to the reaction mixture. The reaction was stirred overnight and insoluble side products were removed from the reaction mixture by filtration, followed by a removal of the solvent under reduced pressure. The ZnPc-NHS (TT1-NHS) ester was dissolved in DMSO (12 mL) and added to a solution of G4-PAMAM (1,4-diaminobutane core) dendrimer (0.50 g) in methanol (12 mL). The reaction was stirred overnight, followed by a removal of insoluble side products by filtration. 2,5-Dioxopyrrolidin-1-yl methyl succinate (525 mg) (G. A. Digenis, B. J. Agha, K. Tsuji, M. Kato, M. Shinogi, J. Med. Chem. 1986, 29, 1468-1476) was added to the solution. The reaction mixture was stirred for four days and afterwards 2-amino-2-hydroxymethyl-propane-1,3-diol (327 mg) was added. Potassium carbonate was added in catalytic amounts to promote the reaction. After four days stirring at ambient temperature, the reaction was quenched with water to hydrolyze the unreacted methyl esters to carboxylic groups. The final compound was purified by dialysis and filtration. After freeze-drying the dendrimer—ZnPc (TT1) nano-system was gained as a dark blue solid (250 mg). The structure was confirmed by NMR (mixed dendrimer surface with 55 Carboxylate and 6 TRIS dendrimer surface groups in average) and the ZnPc (TT1) loading was measured by means of UV/Vis spectroscopy resulting in 3 ZnPc (TT1) molecules per dendrimer in average. The nano-system size was measured by nano-particle tracking analysis (NTA) and found to have 58±4 nm mean hydrodynamic size as shown in Table 1 and FIG. 1(C).

### EXAMPLE 7

Covalent Carboxylate/TRIS G5-PAMAM Dendrimer—ZnPc (TT1) Nano-System, Having an Average of 8 ZnPc (TT1) Molecules Per Dendrimer

[Cov-CTT-G5-PD-(TT1)₈]

ZnPc (TT1, 173 mg) was dissolved in dichloromethane (10 mL). N-Hydroxysuccinimide (25.4 mg) was dissolved in DMSO (10 mL) and added to the ZnPc (TT1) solution followed by the addition of N,N′-Dicyclohexylcarbodiimide (45.2 mg) to the reaction mixture. The reaction was stirred overnight and insoluble side products were removed from the reaction mixture by filtration, followed by a removal of the DCM solvent content under reduced pressure. The ZnPc-NHS (TT1-NHS) ester dissolved in dimethyl sulfoxide (DMSO) was then added to a solution of G5-PAMAM (1,4-diaminobutane core) dendrimer (0.80 g) in dry methanol (25 mL). The reaction was stirred overnight, followed by a removal of insoluble side products by filtration. 2,5-Dioxopyrrolidin-1-yl methyl succinate (844 mg) (G. A. Digenis, B. J. Agha, K. Tsuji, M. Kato, M. Shinogi, J. Med. Chem. 1986, 29, 1468-1476) was added directly to the solution. The reaction mixture was stirred for two days and afterwards 2-amino-2-hydroxymethyl-propane-1,3-diol (516 mg) was added. Potassium carbonate was added in catalytic amounts to promote the reaction. After four days stirring at ambient temperature, the reaction was quenched with water to hydrolyze the unreacted methyl esters to carboxylic groups. The final compound was purified by dialysis and filtration. After freeze-drying the dendrimer—ZnPc (TT1) nano-system was gained as a dark blue solid (420 mg). The structure was confirmed by NMR (mixed dendrimer surface with 113 Carboxylate and 7 TRIS dendrimer surface groups) and the ZnPc (TT1) loading was measured by means of UV/Vis spectroscopy resulting in 8 ZnPc (TT1) molecules per dendrimer in average. The nano-system size was measured by nano-particle tracking analysis (NTA) and found to have 161±29 nm mean hydrodynamic size as shown in Table 1 and FIG. 1(I).

### EXAMPLE 8

Covalent 4-carbomethoxy pyrrolidone G4-PAMAM Dendrimer—ZnPc (TT1) Nano-System, having an Average of 2 ZnPc (TT1) Molecules Per Dendrimer

[Cov-PT-G4-PD-(TT1)₂]

The activated ZnPc-NHS (TT1-NHS) ester was prepared by dissolving ZnPc (TT1, 606 mg) in dichloromethane (30 mL). N-Hydroxysuccinimide (182 mg) was dissolved in DMSO (40 mL) and added to the ZnPc (TT1) solution followed by the addition of N,N′-dicyclohexylcarbodiimide (230 mg) to the reaction mixture. The reaction was stirred overnight and insoluble side products were removed from the reaction mixture by filtration, followed by a removal of the DCM solvent content under reduced pressure. The activated ZnPc-NHS (TT1-NHS) ester dissolved in DMSO was then added to a solution of G4-PAMAM (1,4-diaminobutane core) dendrimer (5.6 g) in methanol (80 mL). The reaction was stirred 2 days, followed by a removal of insoluble side products by filtration. The dendrimer-ZnPc (TT1) solution was then directly used for the dendrimer surface functionalization. The PAMAM dendrimer solution from the previous reaction was taken and added to a solution of dimethyl itaconate (5.2 g) dissolved in methanol (15 mL). The solution was cooled with an ice bath during the addition. The reaction was stirred for four days. The final compound was purified by dialysis and filtration. After freeze-drying the dendrimer—ZnPc (TT1) nano-system was gained as a dark blue solid (6.7 g). The structure was confirmed by NMR and the ZnPc (TT1) loading was measured by means of UV/Vis spectroscopy resulting in 2 ZnPc (TT1) molecules per dendrimer in average. The nano-system size was measured by nano-particle tracking analysis (NTA) and found to have 46±3 nm mean hydrodynamic size as shown in Table 1 and FIG. 1(L).

### EXAMPLE 9

Covalent 4-carbomethoxy pyrrolidone G5-PAMAM Dendrimer—ZnPc (TT1) Nano-System, having an Average of 4 ZnPc (TT1) Molecules Per Dendrimer

[Cov-PT-G5-PD-(TT1)₄]

The activated ZnPc-NHS (TT1-NHS) ester was prepared by dissolving ZnPc (TT1, 239 mg) in dichloromethane (15 mL). N-Hydroxysuccinimide (75 mg) was dissolved in DMSO (25 mL) and added to the ZnPc (TT1) solution followed by the addition of N,N′-dicyclohexylcarbodiimide (86 mg) to the reaction mixture. The reaction was stirred overnight and insoluble side products were removed from the reaction mixture by filtration, followed by a removal of the DCM solvent content under reduced pressure. The activated ZnPc-NHS (TT1-NHS) ester dissolved in DMSO was then added to a solution of G5-PAMAM (1,4-diaminobutane core) dendrimer (2.2 g) in methanol (25 mL). The reaction was stirred 2 days, followed by a removal of insoluble side products by filtration. The dendrimer-ZnPc (TT1) solution was then directly used for the dendrimer surface functionalization. The PAMAM dendrimer solution from the previous reaction was taken and added to a solution of dimethyl itaconate (2.01 g) dissolved in methanol (5 mL). The solution was cooled with an ice bath during the addition. The reaction was stirred for four days. The final compound was purified by dialysis and filtration. After freeze-drying the dendrimer—ZnPc (TT1) nano-system was gained as a dark blue solid (2.7 g). The structure was confirmed by NMR and the ZnPc (TT1) loading was measured by means of UV/Vis spectroscopy resulting in 4 ZnPc (TT1) molecules per dendrimer in average. The nano-system size was measured by nano-particle tracking analysis (NTA) and found to have 100±8 nm mean hydrodynamic size as shown in Table 1 and FIG. 1(K).

### EXAMPLE 10

Covalent Amine G3-PAMAM Dendrimer—ZnPc (TT1) Nano-System, Having an Average of 1.4 ZnPc (TT1) Molecules Per Dendrimer

[Cov-AT-G3-PD-(TT1)_{1.4}]

The activated ZnPc-NHS (TT1-NHS) ester was prepared by dissolving ZnPc (TT1, 17.3 mg) in dichloromethane (1 mL). N-Hydroxysuccinimide (2.54 mg) was dissolved in DMSO (2 mL) and added to the ZnPc (TT1) solution followed by the addition of N,N′-Dicyclohexylcarbodiimide (4.52 mg) to the reaction mixture. The reaction was stirred overnight and insoluble side products were removed from the reaction mixture by filtration, followed by a removal of the DCM solvent content under reduced pressure. The activated ZnPc-NHS (TT1-NHS) ester dissolved in DMSO was added to a solution of G3-PAMAM (1,4-diaminobutane core) dendrimer (100 mg g, 14.7 μmol) in dry methanol (2.5 mL). The reaction was stirred 4 days. The final compound was purified by dialysis and filtration. After freeze-drying the dendrimer—ZnPc (TT1) nano-system was gained as a dark blue solid (93 mg). The structure was confirmed by NMR and the ZnPC (TT1) loading was measured by means of UV/Vis spectroscopy resulting in 1.4 ZnPc (TT1) molecules per dendrimer in average. The nano-system size was measured by nano-particle tracking analysis (NTA) and found to have 95±3 nm mean hydrodynamic size as shown in Table 1 and FIG. 1(D).

### EXAMPLE 11

Non-Covalent 4-carbomethoxy pyrrolidone G4-PAMAM Dendrimer—ZnPc (TT1) Nano-System, Having an Average of 2 ZnPc (TT1) Molecules Per Dendrimer

[NonCov-PT-G4-PD-(TT1)₂]

The 4-Carbomethoxy pyrrolidone G4-PAMAM (1,4-diaminobutane core) dendrimer (106 mg) was dissolved in chloroform (4 mL) and the ZnPc (TT1, 7.5 mg) dissolved in THF (2 mL) was added. The mixture was stirred for 2 h at 40° C. Afterwards the solvent was removed in vacuum. The dark blue compound was then taken up in water and filtered. Freeze-drying of the aqueous solution yielded the dendrimer—ZnPc (TT1) nano-system (103 mg) as a dark blue solid. The ZnPc (TT1) loading was measured by means of UV/Vis spectroscopy resulting in 2 ZnPc (TT1) molecules per dendrimer in average. The nano-system size was measured by nano-particle tracking analysis (NTA) and found to have 86±4 nm mean hydrodynamic size as shown in Table 1 and FIG. 1(E).

### EXAMPLE 12

Non-Covalent 4-carbomethoxy Pyrrolidone G4-PAMAM Dendrimer—ZnPc (TT1) Nano-System, Having an Average of 2 ZnPc (TT1) Molecules Per Dendrimer

[NonCov-PT-G4-PD-(TT1)₂]

The 4-Carbomethoxy pyrrolidone G4-PAMAM dendrimer (1,4-diaminobutane core) (2.12 g) was dissolved in Methanol (50 mL) and the ZnPc (TT1) (150 mg) dissolved in THF (12 mL) was added. The mixture was stirred for 2.5 h at 40° C. The dark blue solution was then added to stirred solution of water (500 mL) and filtered. Freeze-drying of the aqueous solution yielded the dendrimer—ZnPc (TT1) nano-system (1.8 g) as a dark blue solid. The ZnPc (TT1) loading was measured by means of UV/Vis spectroscopy resulting in 2 ZnPc (TT1) molecules per dendrimer in average. The nano-system size was measured by nano-particle tracking analysis (NTA) and found to have 84±5 nm mean hydrodynamic size as shown in Table 1 and FIG. 1(J).

### EXAMPLE 13

Non-Covalent 4-carbomethoxy pyrrolidone G3-PAMAM Dendrimer—ZnPc (TT1) Nano-System, Having an Average of 0.5 ZnPc (TT1) Molecules Per Dendrimer [NonCov-PT-G3-PD-(TT1)_{0.5}]

The 4-Carbomethoxy pyrrolidone G3-PAMAM (1,4-diaminobutane core) dendrimer (319 mg) was dissolved in dichloromethane (15 mL) and the ZnPc (TT1, 45 mg) dissolved in dichloromethane (7 mL) was added. The mixture was stirred for 15 minutes before methanol (45 mL) was added to the mixture. After 20 minutes incubation time, a small amount of water (10 mL) was added and the mixture was stirred for another 15 minutes. The solvent was removed in vacuum until around 5 mL of water were left in the flask. The dark blue liquid was then taken up by adding additional water and was filtered. Freeze-drying of the aqueous solution yielded the dendrimer—ZnPc (TT1) nano-system (324 mg) as a dark blue solid. The ZnPc (TT1) loading was measured by means of UV/Vis spectroscopy resulting in 0.5 ZnPc (TT1) molecules per dendrimer in average. The nano-system size was measured by nano-particle tracking analysis (NTA) and found to have 80±3 nm mean hydrodynamic size as shown in Table 1.

### EXAMPLE 14

Non-Covalent 4-carbomethoxy pyrrolidone G4-PAMAM Dendrimer—ZnPc (TT1) Nano-System, having an Average of 4 ZnPc (TT1) Molecules Per Dendrimer

[NonCov-PT-G4-PD-(TT1)₄]

The 4-Carbomethoxy pyrrolidone G4-PAMAM (1,4-diaminobutane core) dendrimer (636 mg) was dissolved in dichloromethane (50 mL) and the ZnPc (TT1, 90 mg) dissolved in dichloromethane (10 mL) was added. The mixture was stirred for 15 minutes before methanol (100 mL) was added to the mixture. After 20 minutes incubation time, a small amount of water (5 mL) was added and the mixture was stirred for another 15 minutes. The solvent was removed in vacuum. The dark blue compound was then taken up in water and filtered. Freeze-drying of the aqueous solution yielded the dendrimer—ZnPc (TT1) nano-system (586 mg) as a dark blue solid. The ZnPc (TT1) loading was measured by means of UV/Vis spectroscopy resulting in 4 ZnPc (TT1) molecules per dendrimer in average. The nano-system size was measured by nano-particle tracking analysis (NTA) and found to have 191±12 nm mean hydrodynamic size as shown in Table 1 and FIG. 1(H).

### EXAMPLE 15

Non-Covalent 4-carbomethoxy pyrrolidone G5-PAMAM Dendrimer—ZnPc (TT1) Nano-System, Having an Average of 2.3 ZnPc (TT1) Molecules Per Dendrimer [NonCov-PT-G5-PD-(TT1)_{2.3}]

4-Carbomethoxy pyrrolidone G5-PAMAM (1,4-diaminobutane core) dendrimer [PT-G5-PD] (213 mg) was dissolved in dichloromethane (10 mL) and the ZnPc (TT1, 30 mg) dissolved in dichloromethane (5 mL) was added. The mixture was stirred for 15 minutes before methanol (30 mL) was added to the mixture. After 20 minutes incubation time, a small amount of water (5 mL) was added and the mixture was stirred for another 15 minutes. The solvent was removed in vacuum. The dark blue compound was then taken up in water and filtered. Freeze-drying of the aqueous solution yielded the dendrimer—ZnPc (TT1) nano-system (150 mg) as a dark blue solid. The ZnPc (TT1) loading was measured by means of UV/Vis spectroscopy resulting in 2.3 ZnPc (TT1) molecules per dendrimer in average. The nano-system size was measured by nano-particle tracking analysis (NTA) and found to have 136±2 nm mean hydrodynamic size as shown in Table 1.

### EXAMPLE 16

Non-Covalent Carboxylate/TRIS G4-PAMAM Dendrimer—ZnPc (TT1) Nano-System, Having an Average of 3 ZnPc (TT1) Molecules Per Dendrimer

[NonCov-CTT-G4-PD-(TT1)₃]

The Carboxylate/TRIS G4-PAMAM dendrimer (259 mg, 1,4-diaminobutane core, average of 58 Carboxy and 6 TRIS dendrimer surface groups) was dissolved in methanol (25 mL) and the ZnPc (TT1, 39 mg) dissolved in dichloromethane (7 mL) was added. After 20 minutes incubation time, a small amount of water (5 mL) was added and the mixture was stirred for another 15 minutes. The solvent was removed in vacuum. The dark blue compound was then taken up in water and filtered. Freeze-drying of the aqueous solution yielded the dendrimer—ZnPc (TT1) nano-system (252 mg) as a dark blue solid. The ZnPc (TT1) loading was measured by means of UV/Vis spectroscopy resulting in 3 ZnPc (TT1) molecules per dendrimer in average. The nano-system size was measured by nano-particle tracking analysis (NTA) and found to have 62±7 nm mean hydrodynamic size as shown in Table 1 and FIG. 1(F).

### EXAMPLE 17

Non-Covalent Carboxylate/TRIS G5-PAMAM Dendrimer—ZnPc (TT1) Nano-System, Having an Average of 7 ZnPc (TT1) Molecules Per Dendrimer

[NonCov-CTT-G5-PD-(TT1)₇]

The Carboxylate/TRIS G5-PAMAM dendrimer (258 mg, 1,4-diaminobutane core, average of 118 Carboxy and 10 TRIS dendrimer surface groups) was dissolved in methanol (25 mL) and the ZnPc (TT1, 30 mg) dissolved in dichloromethane (7 mL) was added. After 20 minutes incubation time, a small amount of water (5 mL) was added and the mixture was stirred for another 15 minutes. The solvent was removed in vacuum. The dark blue compound was then taken up in water and filtered. Freeze-drying of the aqueous solution yielded the dendrimer—ZnPc (TT1) nano-system (240 mg) as a dark blue solid. The ZnPc (TT1) loading was measured by means of UV/Vis spectroscopy resulting in 7 ZnPc (TT1) molecules per dendrimer in average. The nano-system size was measured by nano-particle tracking analysis (NTA) and found to have 53±5 nm mean hydrodynamic size as shown in Table 1.

### EXAMPLE 18

Non-Covalent Amine G4-PAMAM dendrimer—ZnPc (TT1) Nano-System, Having an Average of 2 ZnPc (TT1) Molecules Per Dendrimer

[NonCov-AT-G4-PD-(TT1)₂]

The amine terminated G5-PAMAM (1,4-diaminobutane core) dendrimer (137 mg) was dissolved in Methanol (8 mL) and the ZnPc (TT1, 7.5 mg) dissolved in THF (2 mL) was added. The mixture was stirred for 2 h at room temperature. Afterwards the solvent was removed in vacuum. The dark blue compound was then taken up in water (12 mL) and. Freeze-drying of the aqueous solution yielded the dendrimer—ZnPc (TT1) nano-system (144 mg) as a dark blue solid. The ZnPc (TT1) loading was measured by means of UV/Vis spectroscopy resulting in 2 ZnPc (TT1) molecules per dendrimer in average.

### EXAMPLE 19

Mixed Covalent and Non-Covalent 4-carbomethoxy pyrrolidone G5-PAMAM Dendrimer—ZnPc (TT1) Nano-System, Having an Average of 4 ZnPc (TT1) Molecules Per Dendrimer Covalently Linked and 2 ZnPc (TT1) Molecules Per Dendrimer Non-Covalently Linked

[Cov/NonCov-PT-G5-PD-(TT1)_{4Covalent}-(TT1)_{2Non-covalent}]

The [Cov-PT-G5-PD-(TT1)₄] (100 mg, Covalent 4-carbomethoxy pyrrolidone G5-PAMAM dendrimer—ZnPc (TT1) nano-system, having an average of 4 ZnPc (TT1) molecules per dendrimer, Example 17) was dissolved in chloroform (4 mL) and the ZnPc (TT1, 3.3 mg) dissolved in THF (2 mL) was added. The mixture was stirred for 2 h at 40° C. Afterwards the solvent was removed in vacuum. The dark blue compound was then taken up in water (12 mL) and filtered. Freeze-drying of the aqueous solution yielded the dendrimer—ZnPc (TT1) nano-system (103 mg) as a dark blue solid. The ZnPc (TT1) loading was measured by means of UV/Vis spectroscopy resulting in 6 ZnPc (TT1) molecules per dendrimer in average [4 ZnPc (TT1) molecules per dendrimer in average covalently linked, and 2 ZnPc (TT1) molecules per dendrimer in average non-covalently linked]. The nano-system size was measured by nano-particle tracking analysis (NTA) and found to have 70±2 nm mean hydrodynamic size as shown in Table 1.

Nano-Systems Size Analysis

Nano-systems size analysis and size distribution profiles were determined by NanoSight LM20 Nano-particle Tracking Analysis (NanoSight, Amesbury, UK) equipped with a sample chamber with a 405 nm blue laser and a Viton fluoroelastomer O-ring. The nano-systems samples were suspended in PBS and then diluted to a suitable concentration for measurement. All measurements were performed at room temperature and repeated at least three times and with different preparations.

The mean hydrodynamic sizes of the nano-systems of Examples 1-17 and 19, determined as described above, are set out in Table 1. The size distribution profiles of twelve of the nano-systems are shown in FIGS. 1(A)-(L), as noted in Table 1. The sizes of the nano-particles present in the nano-systems lie in the range between 1 nm and 500 nm.

TABLE 1

| | Mean | |
|---|---|---|
| | Hydrodynamic | Size Distribution |
| Example | Size (nm) | Figure |
| 1 | 88 ± 15 | 1(A) |
| 2 | 117 ± 14 | — |
| 3 | 91 ± 10 | 1(B) |
| 4 | 86 ± 5 | — |
| 5 | 186 ± 11 | 1(G) |
| 6 | 58 ± 4 | 1(C) |
| 7 | 161 ± 29 | 1(I) |
| 8 | 46 ± 3 | 1(L) |
| 9 | 100 ± 8 | 1(K) |
| 10 | 95 ± 3 | 1(D) |
| 11 | 86 ± 4 | 1(E) |
| 12 | 84 ± 5 | 1(J) |
| 13 | 80 ± 3 | — |
| 14 | 191 ± 12 | 1(H) |
| 15 | 136 ± 2 | — |
| 16 | 62 ± 7 | 1(F) |
| 17 | 53 ± 5 | — |
| 19 | 70 ± 2 | — |

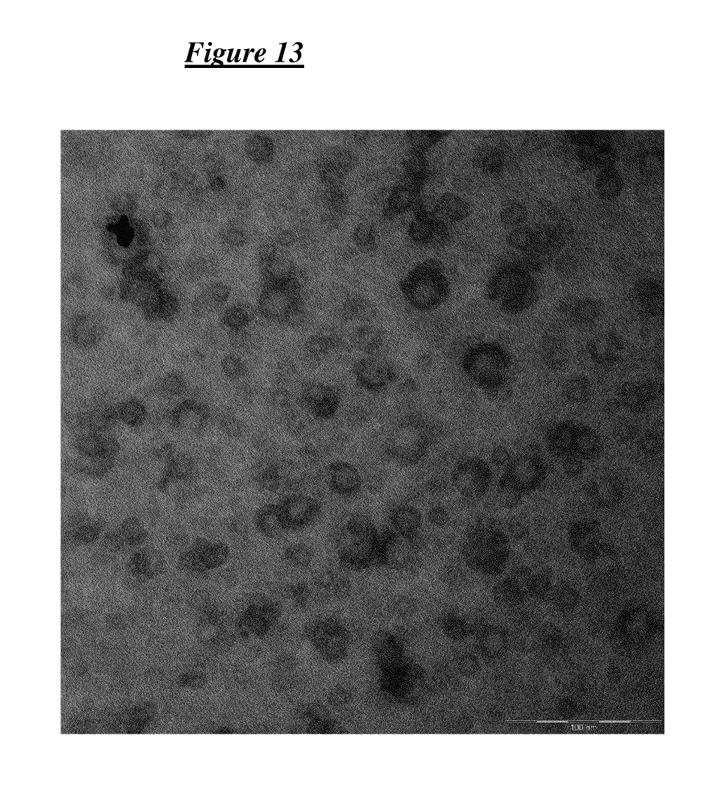

A Transmission Electron Microscopy image of the nano-system of Example 8 is shown in FIG. 13. The minimum size of the nano-particles of that Example is 9.692 nm.

Determination of Complement Activation by the Nano-Systems

To measure complement activation in vitro, the human serum complement products C5a and sC5b-9 were determined using the respective ELISA kits (Quidel, San Diego, Calif., USA) according to the manufacturer's protocols. Before the experiment, the human serum was prepared, characterized and assessed for complement pathways. Briefly, complement activation was initiated by adding the required quantity of nano-system sample to undiluted serum in Eppendorf tubes in a shaking water bath at 37° C. for 30 min, unless stated otherwise. Reactions were terminated by quickly cooling the nano-system samples on ice and adding 25 mM ethylenediaminetetraacetic acid (EDTA). After centrifugation, the supernatant was used for the determination and quantification of complement activation products C5a and sC5b-9. Control plasma incubation contained PBS (the same volume as the nano-system samples) to assess background levels, and zymosan (200 μg/mL) was used as a positive control for monitoring complement activation throughout.

The results are shown in Table 2.

TABLE 2

| | Folds (sC5b-9 | | Folds (C5a | |
|---|---|---|---|---|
| | Concentration | | Concentration | |
| | vs | | vs | |
| Nano-system^{a} | Background) | SD | Background) | SD |
| Background | 1.000 | 0.057 | 1.000 | 0.054 |
| (PBS) | | | | |
| Example 1 | 2.213 | 0.111 | 1.711 | 0.096 |
| Example 2 | 3.306 | 0.165 | 2.241 | 0.112 |
| Example 3 | 1.208 | 0.060 | 1.107 | 0.055 |
| Example 4 | 1.274 | 0.064 | 1.320 | 0.078 |
| Example 5 | 1.135 | 0.057 | 1.235 | 0.062 |
| Example 6 | 0.980 | 0.049 | 0.915 | 0.046 |
| Example 7 | 1.592 | 0.080 | 2.050 | 0.102 |
| Example 8 | 1.146 | 0.057 | 1.336 | 0.107 |
| Example 9 | 1.724 | 0.086 | 1.812 | 0.116 |
| Example 10 | 2.073 | 0.104 | 1.971 | 0.099 |
| Example 11 | 1.216 | 0.061 | 1.206 | 0.060 |
| Example 13 | 1.056 | 0.053 | 1.397 | 0.071 |
| Example 14 | 5.662 | 0.283 | 4.533 | 0.254 |
| Example 15 | 1.529 | 0.076 | 1.093 | 0.055 |
| Example 16 | 1.345 | 0.067 | 1.051 | 0.053 |
| Example 17 | 1.470 | 0.074 | 0.933 | 0.047 |
| Example 19 | 2.541 | 0.127 | 2.541 | 0.140 |
| Zymosan | 21.266 | 1.206 | 13.505 | 0.905 |
| (200 μg/mL) | | | | |

^{a}All nano-system samples were measured at the concentration of 0.144 μM. PBS was used to assess background levels, and zymosan (200 μg/mL) was used as a positive control.

In Vitro Data—Atherosclerosis

The therapeutic efficacy of the nano-systems according to the invention was studied in mouse macrophage cell line RAW 264.7. Macrophages are the most relevant target cell type for studying the efficacy, in the therapy of atherosclerosis, with these nano-systems. The experiments were performed with a custom-made LED device (λ=670 nm) for illuminating cells on a 96-well plate. The illumination time used was 10 minutes with LED light intensity of 3.04 mW/cm². Cells without nano-systems and/or without light activation were always used as a control, as well as control solutions in which the nano-systems were made. For each sample, a wide range of dilution series was tested.

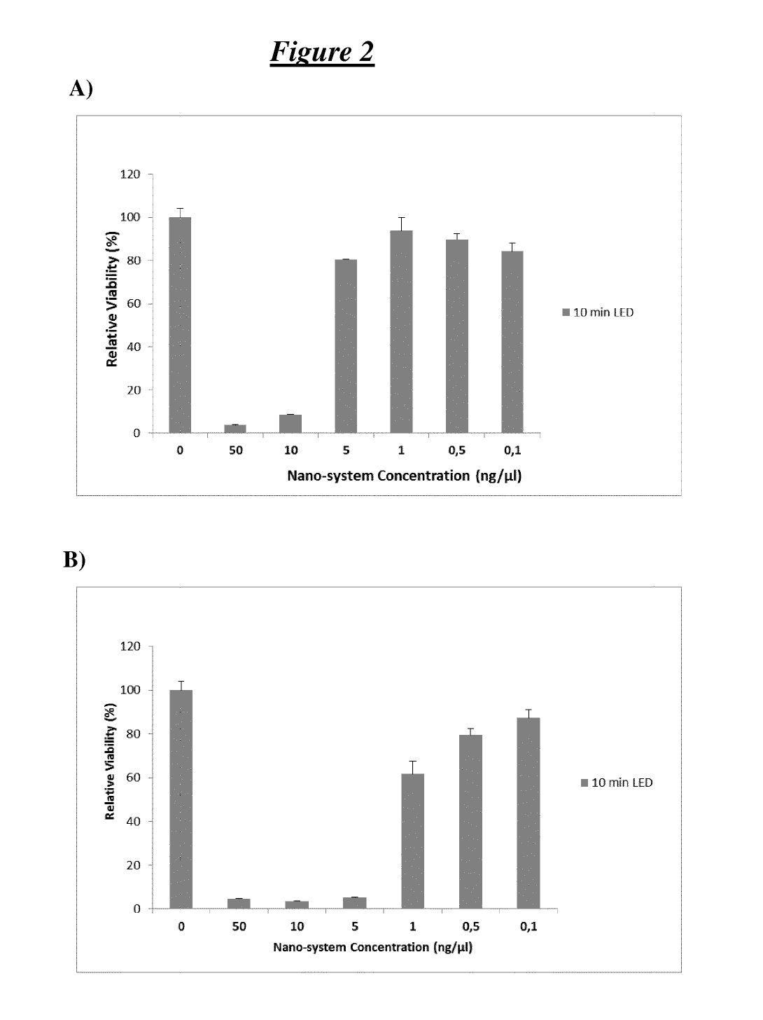

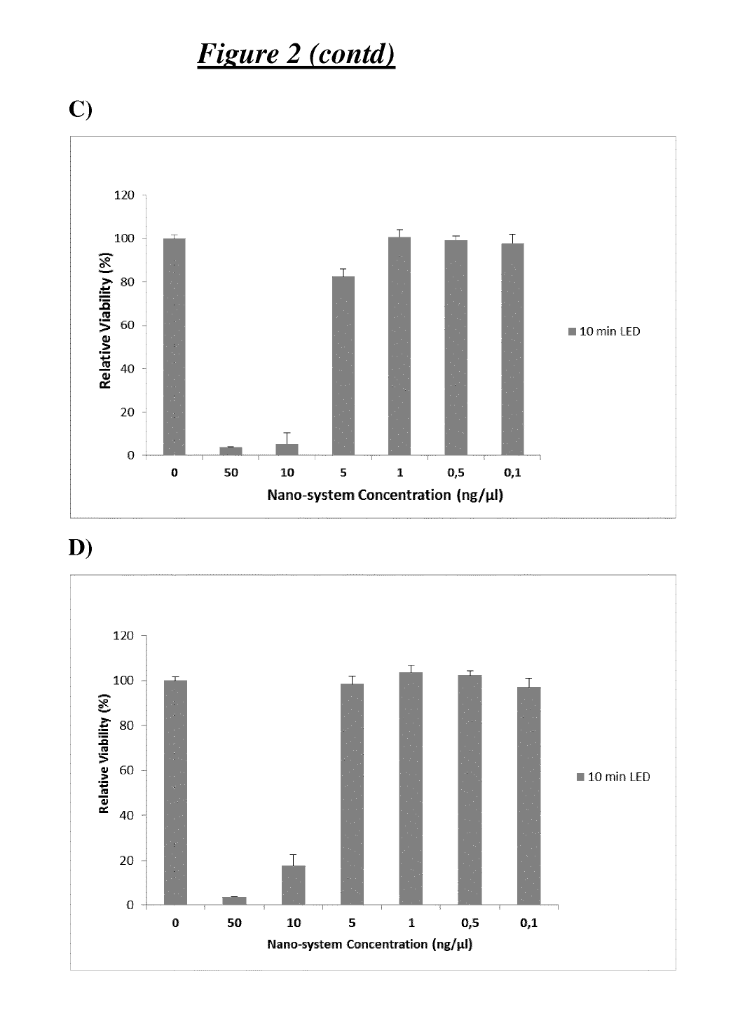

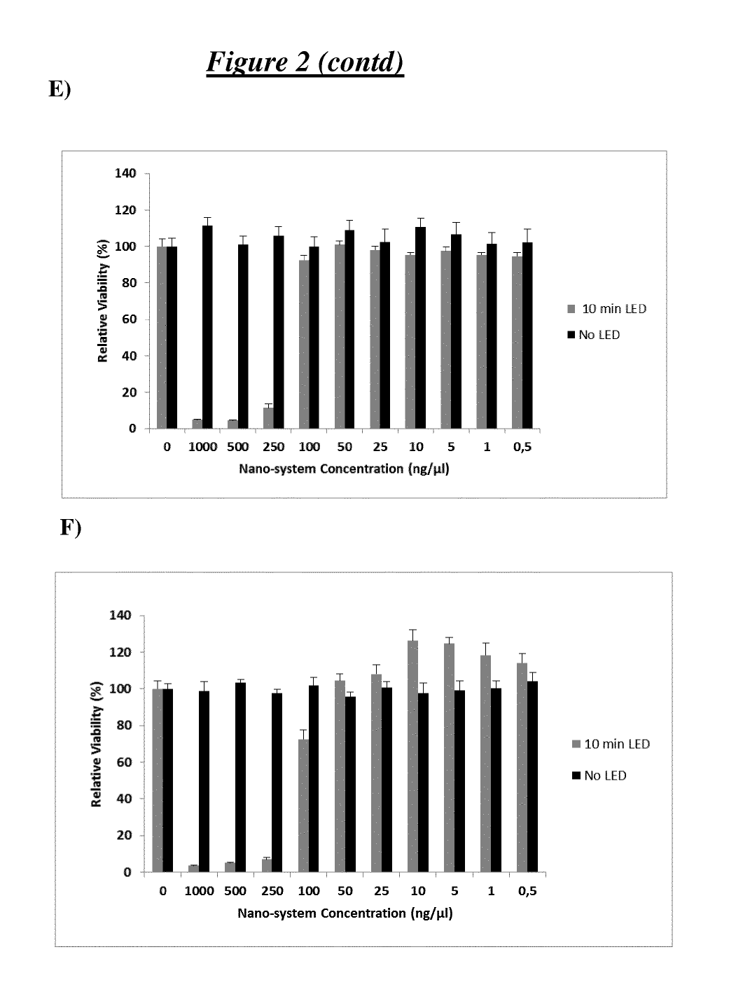

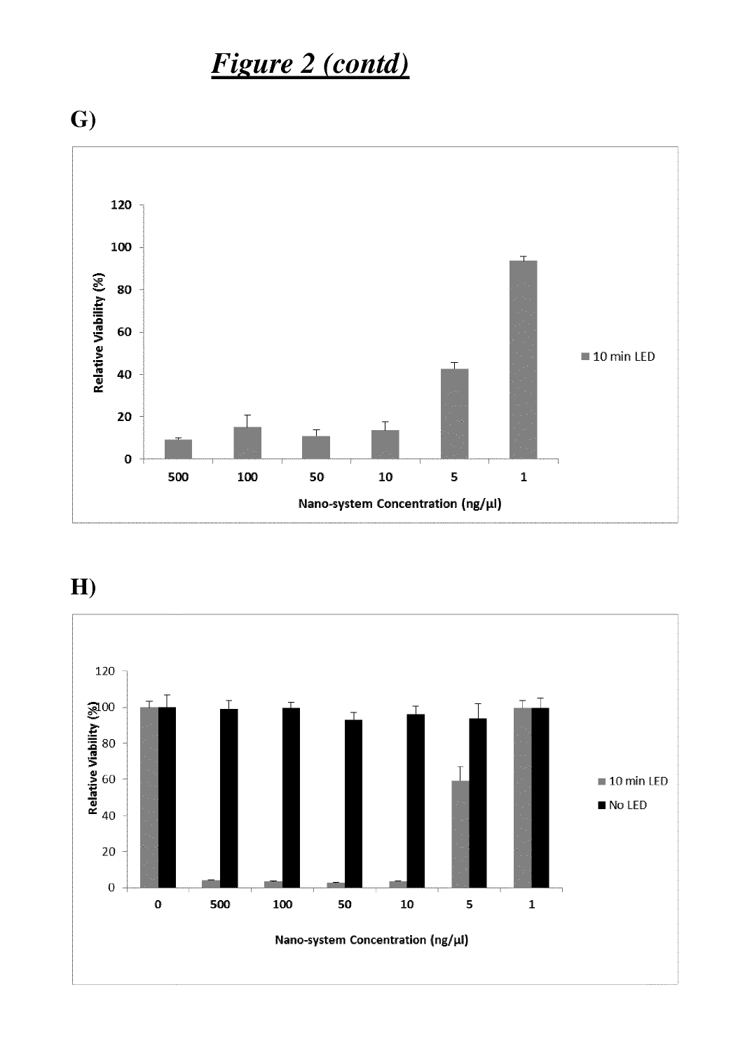

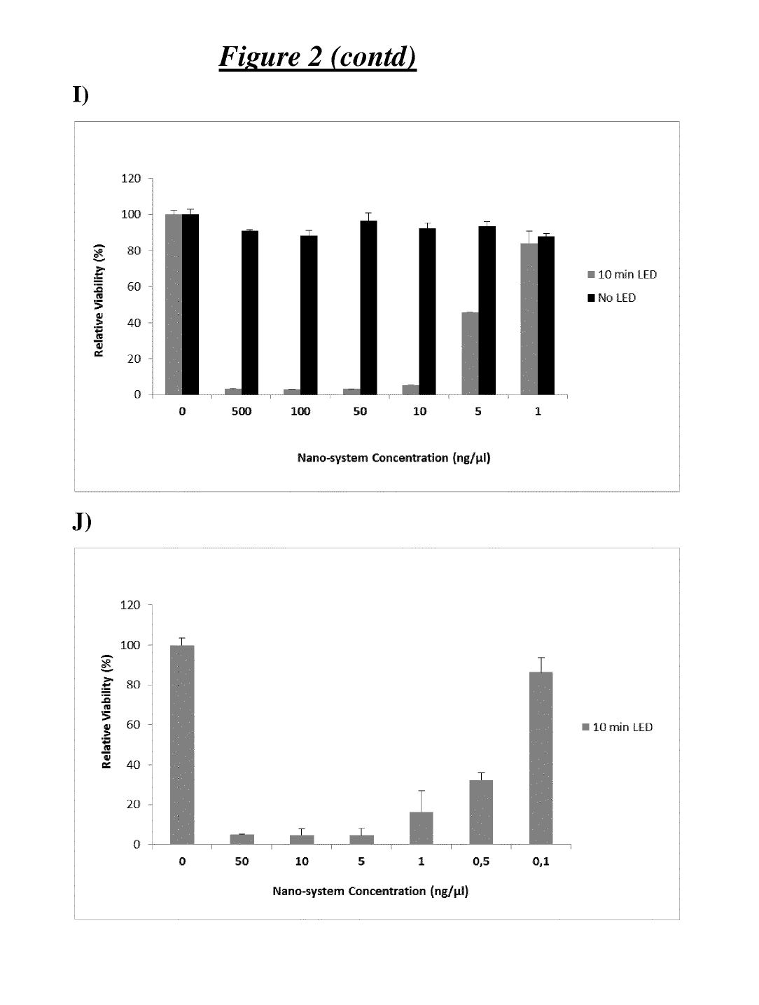

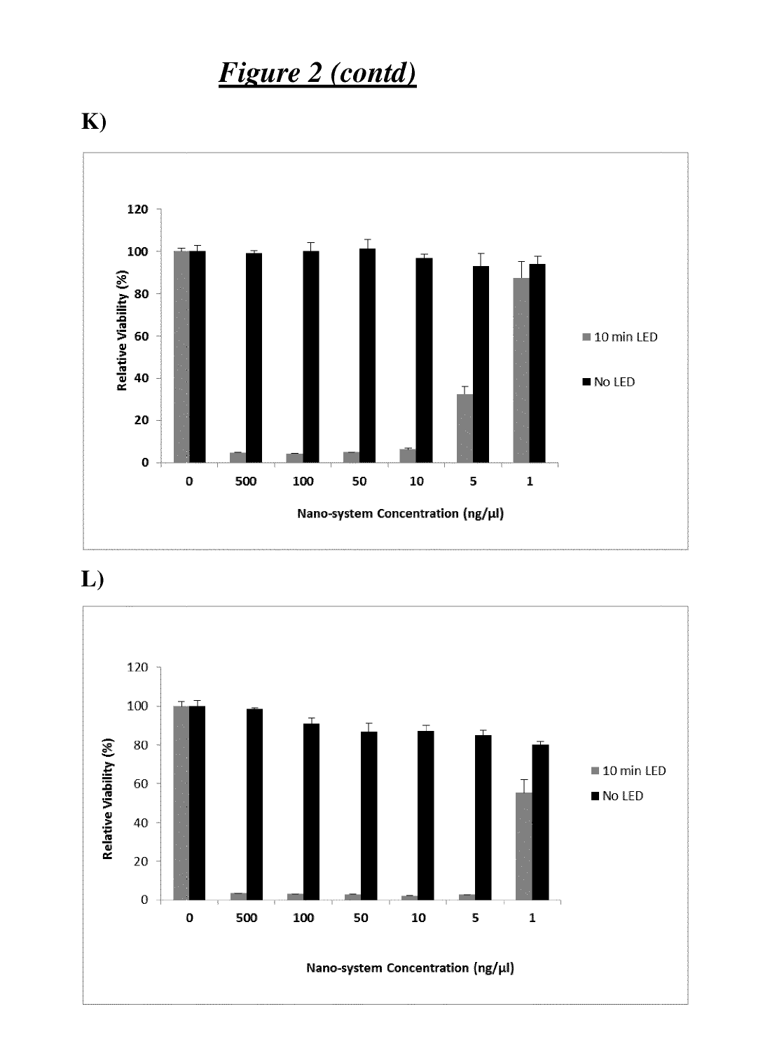

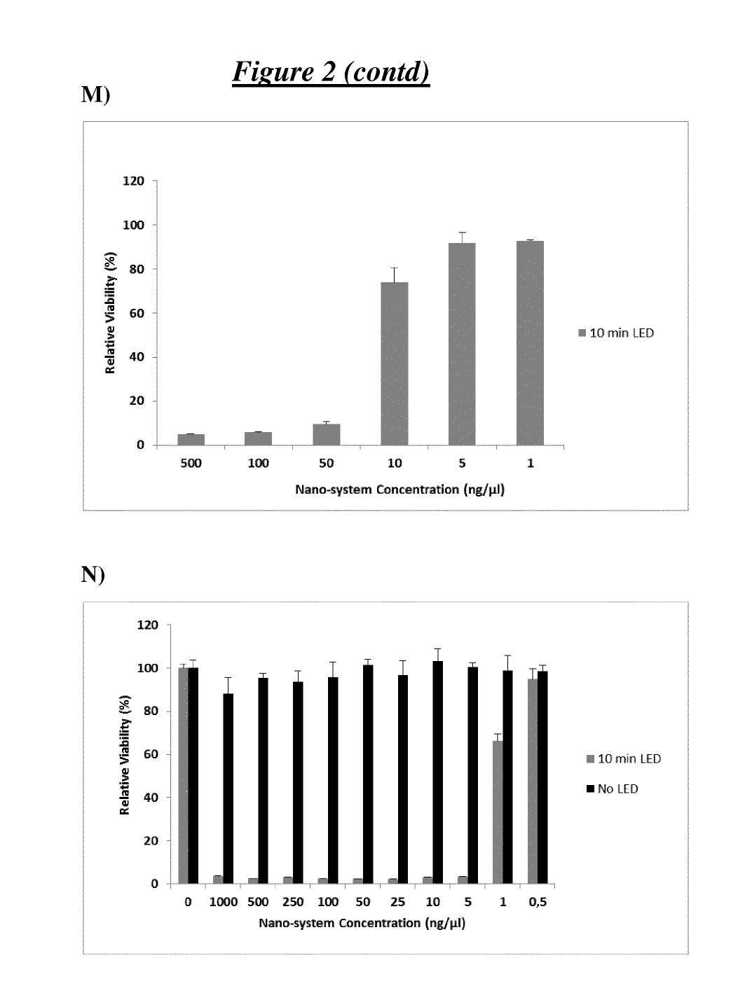

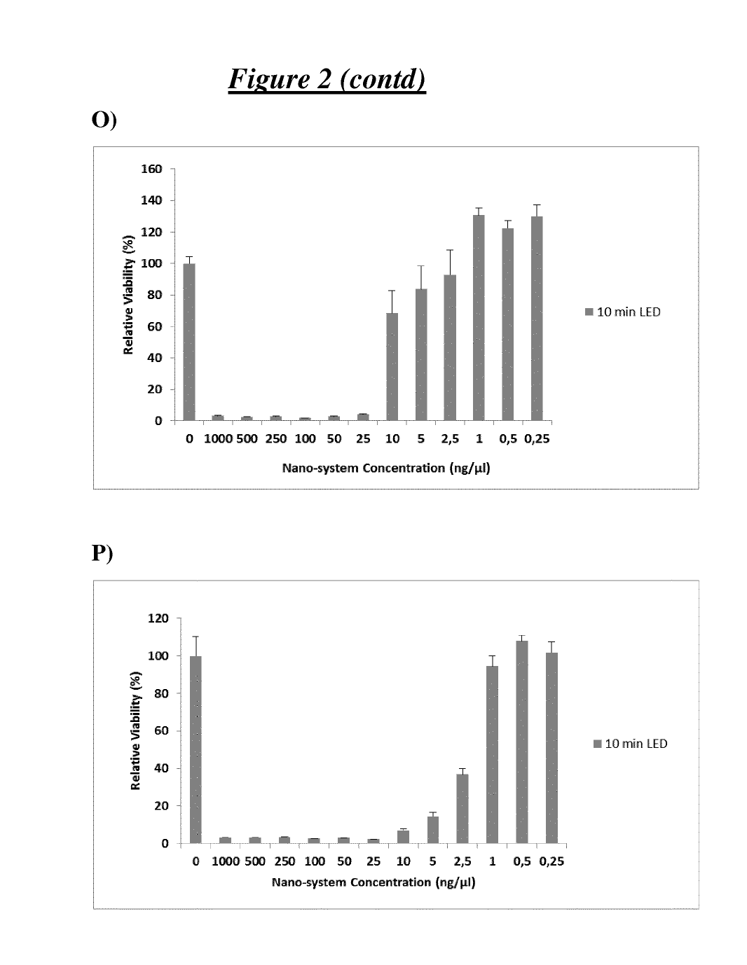

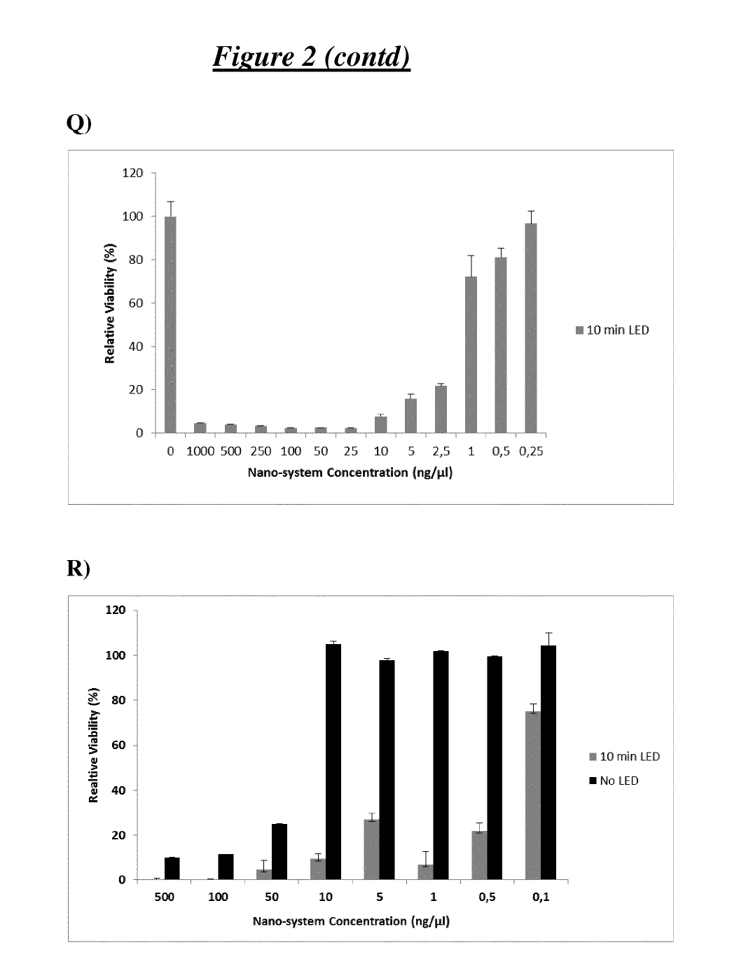

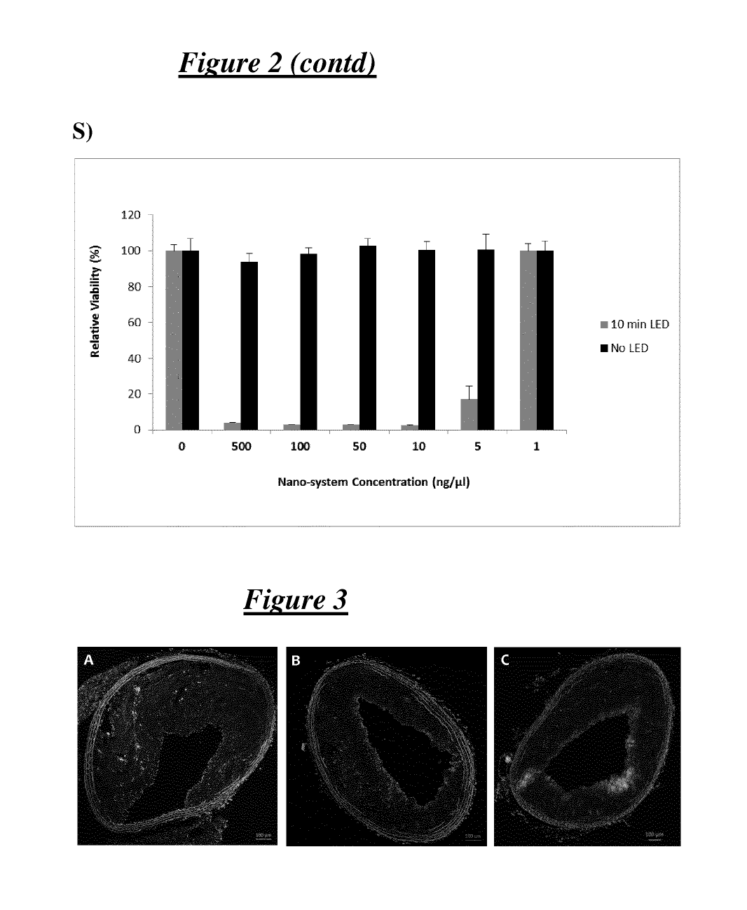

The in vitro therapeutic efficacy of the nano-systems according to Examples 1 to 19 is illustrated in FIGS. 2A-S.

The IC50 values in RAW cells of each of the nano-systems according to Examples 1 to 19 are set out in Table 3:

TABLE 3

| Example | IC50 (ng/μL) |
|---|---|
| 1 | 7 |
| 2 | 2 |
| 3 | 7 |
| 4 | 8 |
| 5 | 150 |
| 6 | 150 |
| 7 | 5 |
| 8 | 6 |
| 9 | 5 |
| 10 | 0.4 |
| 11 | 3 |
| 12 | 1.5 |
| 13 | 25 |
| 14 | 1.5 |
| 15 | 15 |
| 16 | 2.5 |
| 17 | 2 |
| 18 | 0.3 |
| 19 | 3 |

In Vitro Data—Cancer

In order to determine if nano-systems according to the invention are phototoxic to cancer cells, an in vitro experiment similar to that described above, but using breast cancer cell line MCF-7, was performed, using the nano-system of Example 11.

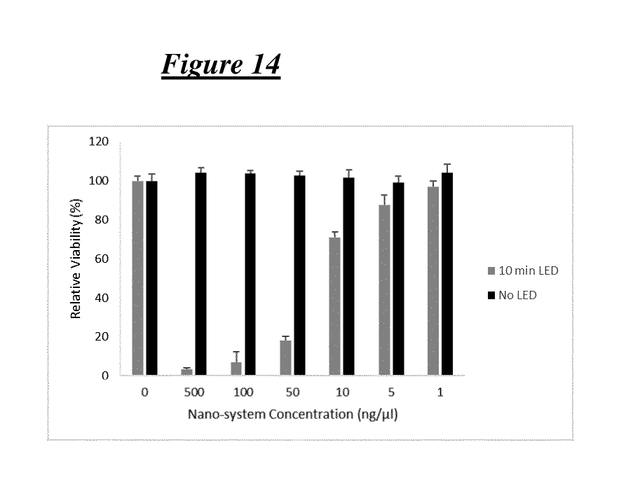

The results are shown in FIG. 14. The IC50 value in MCF-7 cells of the nano-system of Example 11 is 30 ng/μL.

In Vivo Data

a) In Vivo Accumulation of a Nano-System in Mouse Atherosclerotic Plaques

Nano-systems according to Example 14, Example 13, and Example 7 were tested in a LDLR^{-/-}/ApoB^{100/100} mouse model of atherosclerosis. Atherosclerotic plaques were analyzed by fluorescence microscopy. As shown in FIG. 3C, a nano-system according to Example 7 accumulates inside a mouse atherosclerotic plaque and shows the strongest fluorescence signal. As shown in FIG. 3A, a nano-system according to Example 14 shows some fluorescence signal in a mouse atherosclerotic plaque, whereas with a nano-system according to Example 13 the fluorescence signal is hardly visible in a mouse atherosclerotic plaque, as shown in FIG. 3B.

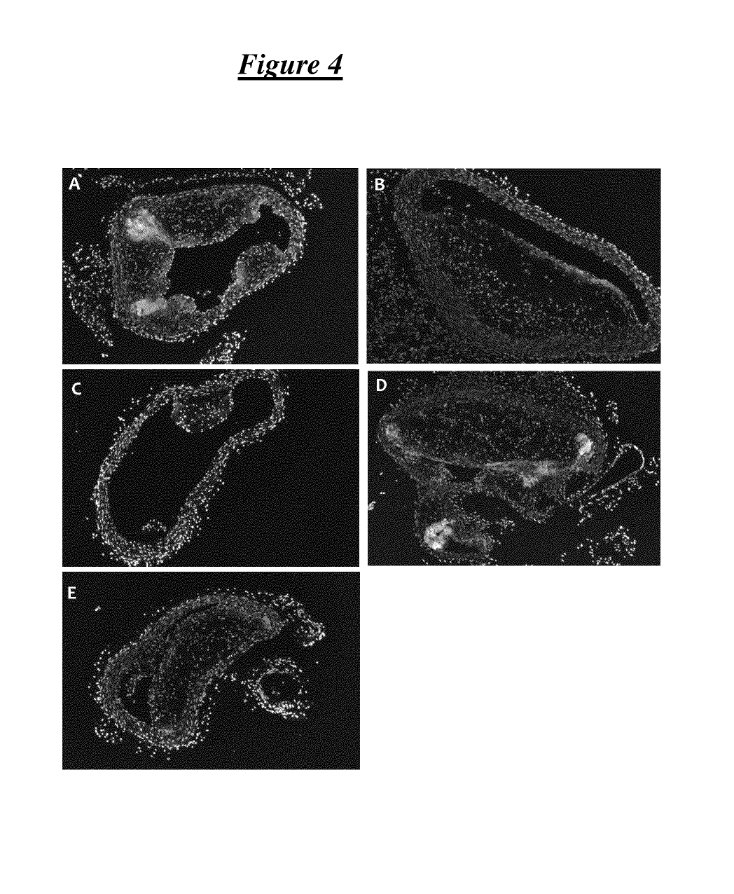

Nano-systems according to Example 4, Example 1, Example 3, Example 8, and Example 9 were tested in a LDLR^{-/-}/ApoB^{100/100} mouse model of atherosclerosis. Atherosclerotic plaques were analyzed by fluorescence microscopy, and the images shown in FIGS. 4A-E show the accumulation of a nano-system according to Example 4, Example 1, Example 3, Example 8, and Example 9 respectively inside an atherosclerotic plaque of a LDLR^{-/-}/ApoB^{100/100} mouse model of atherosclerosis.

b) In Vivo Accumulation of a Nano-System in Rabbit Atherosclerotic Plaques

Nano-systems according to Example 14, Example 13, and Example 7 were tested in a balloon-injured New Zealand White (NZW) rabbit model of atherosclerosis.

Atherosclerotic plaques were analyzed by fluorescence microscopy. As shown in FIG. 5C, a nano-system according to Example 7 accumulates inside a rabbit atherosclerotic plaque and shows the strongest fluorescence signal. As shown in FIG. 5A, a nano-system according to Example 14 shows a relatively high fluorescence signal in a rabbit atherosclerotic plaque. The fluorescence signal from a nano-system according to Example 13 is the lowest in a rabbit atherosclerotic plaque, as shown in FIG. 5B.

Nano-systems according to Example 1, Example 3, Example 8, and Example 9 were tested in a balloon-injured New Zealand White (NZW) rabbit model of atherosclerosis. Atherosclerotic plaques were analyzed by fluorescence microscopy, and the images shown in FIGS. 6A-D show the accumulation of a nano-system according to Example 1, Example 3, Example 8, and Example 9 respectively inside an atherosclerotic plaque of a balloon-injured NZW rabbit model of atherosclerosis.

c) In Vivo Co-Localization of a Nano-System with the Foam/Macrophage Cells in Rabbit Atherosclerotic Plaques

To further verify to which areas in an atherosclerotic plaque a nano-system is accumulating, RAM-11 staining (Foam/Macrophage Cells) and α-SMA staining (Smooth Muscle Cells) were performed. As shown in FIGS. 7A-D, a nano-system according to Example 1 co-localizes with the Foam/Macrophage Cells of an atherosclerotic plaque in a balloon-injured New Zealand White (NZW) rabbit model of atherosclerosis.

d) In Vivo Targeted Accumulation of a Nano-System in Rabbit Atherosclerotic Plaques

To further verify to which areas in an atherosclerotic plaque a nano-system is accumulating, CD31 staining (Endothelium) was performed. As shown in FIG. 8, a nano-system according to Example 8 accumulated in the atherosclerotic plaques in a balloon-injured New Zealand White (NZW) rabbit model of atherosclerosis, and such a nano-system does not accumulate in the endothelium or in the media (smooth muscle cells) or in the adventitia of the arterial wall. Notably, such preferential accumulation in the atherosclerotic plaques occurred despite the absence of any tissue/cell-targeting moiety conjugated to the nano-particles.

Furthermore, a nano-system according to Example 4 does not accumulate in the healthy arterial wall in a balloon-injured New Zealand White (NZW) rabbit model of atherosclerosis, as demonstrated in FIG. 9.

e) Endovascular Targeted Near-Infrared nanoPhotodynamic Therapy (Endovascular Targeted NIR nanoPDT) using a Nano-System which Accumulates in Rabbit Atherosclerotic Plaques

To assess the in vivo therapy effect of the nano-system according to Example 8, which preferentially accumulates in rabbit atherosclerotic plaques without requiring any conjugation of tissue/cell-targeting moieties, aortic cross sections from treated and from non-treated balloon-injured New Zealand White (NZW) rabbit model of atherosclerosis were stained with RAM-11 Foam/Macrophage Cells marker. As shown in FIGS. 10A and B, there was a substantial decrease of the intraplaque Foam/Macrophage Cells in the treated rabbit atherosclerotic plaques compared to the non-treated rabbit atherosclerotic plaques. A close-up of the treated and non-treated atherosclerotic plaques is shown in FIGS. 11A and B.

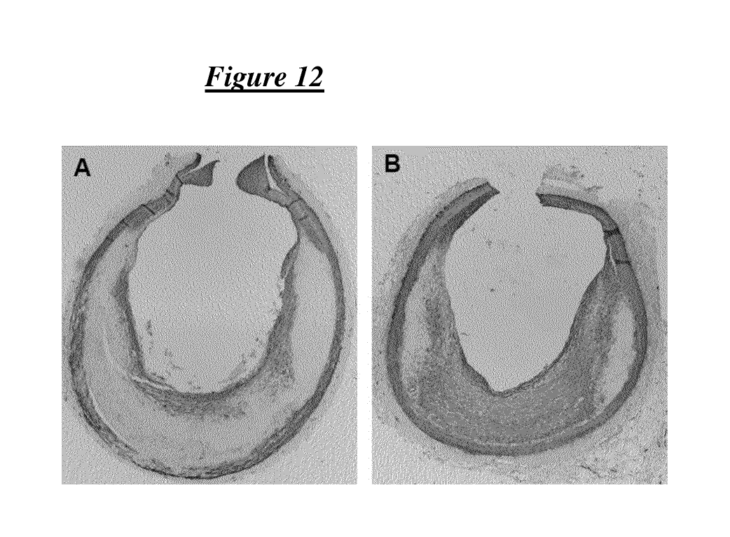

To further assess the in vivo therapy effect of the nano-system according to Example 8, which preferentially accumulates in rabbit atherosclerotic plaques without requiring any conjugation of tissue/cell-targeting moieties, aortic cross sections from treated and from non-treated balloon-injured New Zealand White (NZW) rabbit model of atherosclerosis were stained with a-SMA Smooth Muscle Cells (SMC) marker. As shown in FIGS. 12A and B, there was a substantial increase of the intraplaque Synthetic Smooth Muscle Cells in a layer-structure arrangement in the treated rabbit atherosclerotic plaques compared to the non-treated rabbit atherosclerotic plaques.

f) In Vivo Accumulation of a Nano-System in Inflamed NZW-Rabbit Skeletal Muscle and Skin and Co-Localization with Macrophage Cells

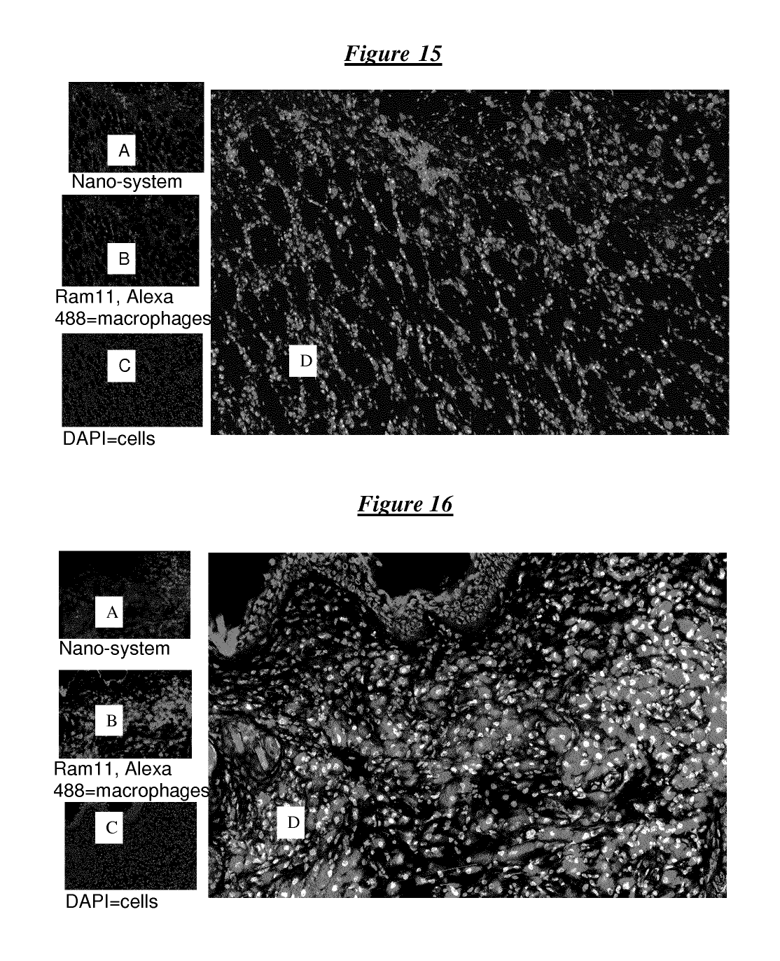

By methods similar to those described above, the nano-system of Example 8 accumulated in inflamed skeletal muscle and in inflamed skin of a NZW-rabbit. The nano-system was co-localized with macrophage cells in the inflamed skeletal muscle—see FIG. 15—and in the inflamed skin—see FIG. 16—again without the presence of any tissue/cell-targeting moieties.

1. A composition comprising self-assembled nano-particles, the nano-particles comprising dendrimers having a phthalocyanine covalently bound to the periphery thereof.

2. A composition as claimed in claim 1, wherein the dendrimers are selected from the group consisting of polyamidoamine (PAMAM) dendrimers, polypropyleneimine (PPI) dendrimers, poly-lysine dendrimers, phosphorus dendrimers and polyester dendrimers.

3. A composition as claimed in claim 1, wherein the dendrimers are dendrimers of the first generation or higher, or the dendrimers are dendrimers of the third generation or higher.

4. A composition as claimed in claim 1, wherein the periphery of the dendrimer is functionalized with one or more surface chemical groups selected from the group consisting of amine, amide, carboxybetaine, sulfobetaine, triazoliumcarboxylate, phosphorylcholine, pyrrolidone, 2-amino-2-hydroxymethyl-propane-1,3-diol, hydroxyl, carboxyl, methoxy, ethoxy, 4-carbomethoxy pyrrolidone, poly(ethylene glycol), and any combination thereof.

5. A composition as claimed in claim 1, wherein the phthalocyanine is a peripherally-substituted phthalocyanine.

6. A composition as claimed in claim 1, wherein the phthalocyanine is a zinc phthalocyanine.

7. A composition as claimed in claim 6, wherein the phthalocyanine is a compound of the formula: 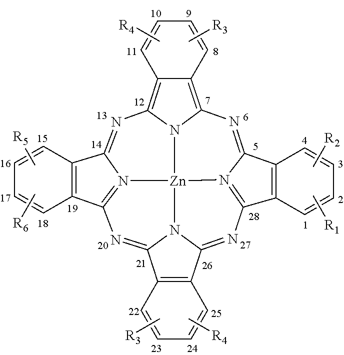
wherein
R₁ and R₂ are independently selected from the group consisting of 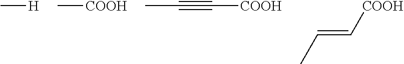
and
R₃, R₄, R₅ and R₆ groups are independently H, an alkyl group having from 1 to 12 carbon atoms, —OR₇, —SR₇ or —NR₇R₈,
in which R₇ and R₈ independently represent H, an alkyl group having from 1 to 12 carbon atoms, or a phenyl group optionally substituted by one or more R₉ groups independently selected from the group consisting of an alkyl group having from 1 to 12 carbon atoms, —OR₁₀, —SR₁₀, and —NR₁₁R₁₂,
wherein R₁₀, R₁₁ and R₁₂ each independently represent H or an alkyl group having from 1 to 12 carbon atoms.

8. A composition as claimed in claim 7, wherein the phthalocyanine is a compound of the formula: 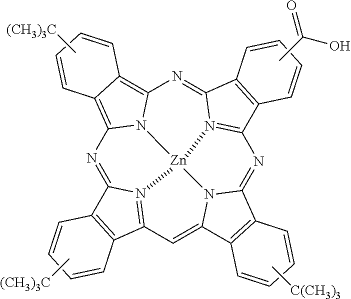
its regioisomers and mixtures thereof, more particularly wherein the phthalocyanine is a compound selected from the group of regioisomers consisting of:
9,16,23-tri-tert-butyl-2-carboxy-5,28:14,19-diimino-7,12:21,26-dinitrilo-tetrabenzo[c,h,m,r][1,6,11,16]tetraazacycloeicosinato-(2⁻)-N²⁹,N³⁰,N³¹,N³² zinc (II);
9,16,24-tri-tert-butyl-2-carboxy-5,28:14,19-diimino-7,12:21,26-dinitrilo-tetrabenzo[c,h,m,r][1,6,11,16]tetraazacycloeicosinato-(2⁻)-N²⁹,N³⁰,N³¹,N³² zinc (II);
9,17,23-tri-tert-butyl-2-carboxy-5,28:14,19-diimino-7,12:21,26-dinitrilo-tetrabenzo[c,h,m,r][1,6,11,16]tetraazacycloeicosinato-(2⁻)-N²⁹,N³⁰,N³¹,N³² zinc (II);
9.17.24-tri-tert-utyl-2-carboxy-5,28:14,19-diimino-7,12:21,26-dinitrilo-tetrabenzo[c,h,m,r][1,6,11,16]tetraazacycloeicosinato-(2⁻)-N²⁹,N³⁰,N³¹,N³² zinc (II);
10,16,23-tri-tert-butyl-2-carboxy-5,28:14,19-diimino-7,12:21,26-dinitrilo-tetrabenzo[c,h,m,r][1,6,11,16]tetraazacycloeicosinato-(2⁻)-N²⁹,N³⁰,N³¹,N³² zinc (II);
10,16,24-tri-tert-butyl-2-carboxy-5,28:14,19-diimino-7,12:21,26-dinitrilo-tetrabenzo[c,h,m,r][1,6,11,16]tetraazacycloeicosinato-(2⁻)-N²⁹,N³⁰,N³¹,N³² zinc (II);
10,17,23-tri-tert-butyl-2-carboxy-5,28:14,19-diimino-7,12:21,26-dinitrilo-tetrabenzo[c,h,m,r][1,6,11,16]tetraazacycloeicosinato-(2⁻)-N²⁹, N³⁰, N³¹, N³² zinc (II);
10,17,24-tri-tert-butyl-2-carboxy-5,28:14,19-diimino-7,12:21,26-dinitrilo-tetrabenzo[c,h,m,r][1,6,11,16]tetraazacycloeicosinato-(2⁻)-N²⁹,N³⁰,N³¹,N³² zinc (II);
and mixtures thereof,
and most particularly wherein the phthalocyanine is a compound comprising a mixture of regioisomers 9(10), 16(17), 23(24)-tri-tent-butyl-2-carboxy-5,28:14,19-diimino-7,12:21,26-dinitrilo-tetra-benzo[c,h,m,r] [1,6,11,16]tetraazacycloeicosinato-(2⁻)-N²⁹,N³⁰,N³¹,N³² zinc (II).

9. A composition as claimed in claim 1, which is polydisperse, comprising a mixture of assemblies of dendrimer dimers, trimers and/or higher multimers, the self-assembled nano-particles having a mean hydrodynamic size in the range 20 to 200 nm.

10. A composition as claimed in claim 1, wherein the nano-particles are at least 5 nm in size.

11. A composition as claimed in claim 1, further comprising a phthalocyanine non-covalently associated with the self-assembled nano-particles.

12. A composition as claimed in claim 1, wherein the nano-particles are not conjugated to tissue/cell-targeting moieties.

13. A composition as claimed in claim 1, wherein the self-assembled nano-particles, following exposure to electromagnetic radiation, produce one or more of fluorescence, reactive oxygen species, heat, an optical signal and an acoustic signal.

14. A composition as claimed in claim 1, in a form suitable for injection, such as a solution or dispersion of the self-assembled nano-particles in an aqueous medium, or a lyophilized material, or a form suitable for topical administration, such as a gel, cream or ointment, or a form suitable for oral administration, such as a tablet or capsule, or a form suitable for direct administration to a lesion of a tissue.

15. A composition as claimed in claim 1, for use in therapy and/or diagnosis and/or therapy monitoring and/or theranostics of a lesion of a tissue, for example an atherosclerotic plaque, an inflammatory lesion, or a tumour.

16. The composition of claim 1, wherein the dendrimers are PAMAM dendrimers.
